(19) United States
(12) Patent Application Publication
(10) Pub. No.: US 2009/0105268 A1
(43) Pub. Date: Apr. 23, 2009

(54) Dihydropyrimidone Multimers and Their Use as Human Neutrophil, Elastase Inhibitors

(76) Inventors: Harry Finch, Essex (GB); Christine Edwards, Essex (GB); Nicholas Charles Ray, Essex (GB); Mary Frances Fitzgerald, Essex (GB)

Correspondence Address: SALIWANCHIK LLOYD & SALIWANCHIK;A PROFESSIONAL ASSOCIATION, PO Box 142950, GAINESVILLE, FL, 32614, US

(21) Appl. No.: 11/993,699

(22) PCT Filed: Jun. 26, 2006
(86) PCT No.: PCT/GB2006/002337

§ 371 Date: Dec. 23, 2008

(30) Foreign Application Priority Data
Jun. 24, 2005 (GB) 0512940.8

(51) Int. Cl. A61K 31/497; C07D 403/12; A61P 11/00

(52) U.S. Cl. 514/252.14; 544/296; 514/256

(57) ABSTRACT

A compound of formula M-L-M (I) wherein L is a linker and each M is independently a group of formula (II) is useful in therapy, e.g. of respiratory diseases.

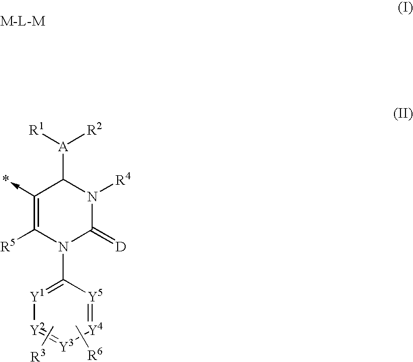

### FIELD OF THE INVENTION

This invention relates to heterocyclic compounds and their use in therapy.

### BACKGROUND TO THE INVENTION

Human neutrophil elastase is a 32 kDa serine proteinase found in the azurophilic granules of neutrophils. It has a role in the degradation of a wide range of extracellular matrix proteins, including fibronectin, laminin, proteoglycans, Type III and Type IV collagens as well as elastin (Bieth, G. In Regulation of Matrix accumulation, Mecham, R. P. (Eds), Academic Press, NY, USA 1986, 217-306). HNE has long been considered to play an important role in homeostasis through repair and disposal of damaged tissues via degradation of the tissue structural proteins. It is also relevant in the defence against bacterial invasion by means of degradation of the bacterial body. In addition to its effects on matrix tissues, HNE has been implicated in the upregulation of IL-8 gene expression and also induces IL-8 release from the epithelial cells of the lung. In animal models of Chronic Obstructive Pulmonary Disease induced by tobacco smoke exposure both small molecule inhibitors and protein inhibitors of HNE inhibit the inflammatory response and the development of emphysema (Wright, J. L. et al. Am. J. Respir. Crit. Care Med. 2002, 166, 954-960; Churg, A. et al. Am. J. Respir. Crit. Care Med. 2003, 168, 199-207). Thus, HNE may play a role both in matrix destruction and in amplifying inflammatory responses in chronic respiratory diseases where neutrophil influx is a characteristic feature. Indeed, HNE is believed to play a role in several pulmonary diseases, including chronic obstructive pulmonary disease (COPD), cystic fibrosis (CF), acute respiratory distress syndrome (ARDS), pulmonary emphysema, pneumonia, severe asthma, sarcoidosis, bronchiectasis and lung fibrosis. It is also implicated in several cardiovascular diseases in which tissue remodelling is involved, for example, in heart failure and the generation of ischaemic tissue injury following acute myocardial infarction. Elevated HNE levels are also correlated with the severity of inflammation in inflammatory bowel disease (Silberer H et al, Clin Lab. 2005; 51 (3-4):117-26) and may play a role in impaired mucosal repair in patients with ulcerative colitis.

COPD is an umbrella term encompassing three different pathological conditions, all of which contribute to limitation of airflow: chronic bronchitis, emphysema and small-airway disease. Generally all three will exist to varying extents in patients presenting with COPD, and all three may be due to neutrophil-mediated inflammation, as supported by the increased number of neutrophils observed in bronchoalveolar leakage (BAL) fluids of COPD patients (Thompson, A. B.; Daughton, D.; et al. Am. Rev. Respir. Dis. 1989, 140, 1527-1537). The major pathogenic determinant in COPD has long been considered to be the protease-anti-protease balance (also known as the ‘elastase:anti-elastase hypothesis’), in which an imbalance of HNE and endogenous antiproteases such as α1-antitrypsin (α₁-AT), Secretory leukocyte protease inhibitor (SLPI) and pre-elafin leads to the various inflammatory disorders of COPD. Individuals that have a genetic deficiency of the protease inhibitor α1-antitrypsin (α1-AT) develop emphysema that increases in severity over-time (Laurrell, C. B.; Erikkson, S Scand. J. Clin. Invest. 1963 15, 132-140). An excess of HNE is therefore destructive, leading to the breakdown of pulmonary morphology with loss of elasticity and destruction of alveolar attachments of airways in the lung (emphysema) whilst simultaneously increasing microvascular permeability and mucus hypersecretion (chronic bronchitis).

Multimeric ligands consist of multiple binding domains which are tethered together through a suitable scaffold. Hence individual binding domains are linked together into a single molecule, increasing the probability that the multimer will bind simultaneously with multiple active sites resulting in high-affinity interactions (Handl, H. L. et al. Expert Opin. Ther. Targets 2004, 8, 565-586; Han, Y. F. et al., Bioorg. Med. Chem. Letts. 1999, 7, 2569-2575). Also, multiple binding interactions with relatively high off-rates can combine to yield an overall low off-rate for the multimeric ligand. Thus, a molecule consisting of a suitable linker and ligands may be expected to show advantage over the monomeric ligands alone in terms of potency and/or duration of action. Multimeric compounds are unlikely to be orally bioavailable (as predicted by Lipinski's “Rule of 5”) which may be advantageous where an inhaled route of administration to the lungs is targeted, since even after inhaled administration, a large proportion of drug is likely to enter the GI tract. Thus such compounds may be expected to show reduced systemic exposure after inhalation administration and hence an improved toxicity profile over orally administered therapies.

Monomers of formula (II) are described as inhibitors of human neutrophil elastase in WO2004/024700, WO2004/024701, GB2392910, WO2005/082863 and WO2005/082864.

### SUMMARY OF THE INVENTION

A first aspect of the invention is a compound of formula (I) or formula (IV):

(M)-(L)-(M) (I)

[(M)-(L)]ₜ-G (IV)

wherein
each M is independently an inhibitor of HNE; and
each L is independently a linker group;
t is 2 to 20; and
G is aryl, heteroaryl, alkyl, cycloalkyl, nitrogen, a dendrimer or a group of any of formulae (V) to (VI):

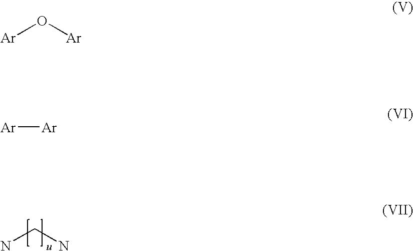

wherein
   Ar is aryl or heteroaryl; and
   u is 2-20;
      M is a group of Formula (II)

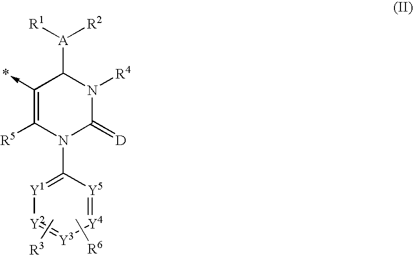

wherein

A is aryl or heteroaryl;

D is oxygen or sulphur;

R¹, R² and R³ are independently each hydrogen, halogen, nitro, cyano, alkyl, hydroxy or alkoxy, wherein alkyl and alkoxy can be further substituted with one to three identical or different radicals selected from the group consisting of halogen, hydroxy and alkoxy;

R⁴ is hydrogen, alkyl, alkylcarbonyl, alkoxycarbonyl, alkenoxycarbonyl, hydroxycarbonyl, aminocarbonyl, arylcarbonyl, heteroarylcarbonyl, heterocycloalkylcarbonyl, heteroaryl, heterocycloalkyl or cyano, wherein alkylcarbonyl, alkoxycarbonyl, and aminocarbonyl can be further substituted with one to three identical or different radicals selected from the group consisting of cycloalkyl, hydroxy, alkoxy, alkoxycarbonyl, hydroxycarbonyl, aminocarbonyl, cyano, amino, heteroaryl, heterocycloalkyl and tri-(alkyl)-silyl, and wherein heteroarylcarbonyl, heterocycloalkylcarbonyl, heteroaryl and heterocycloalkyl can be further substituted with alkyl; or

R⁴ represents a group of Formula (VIII)

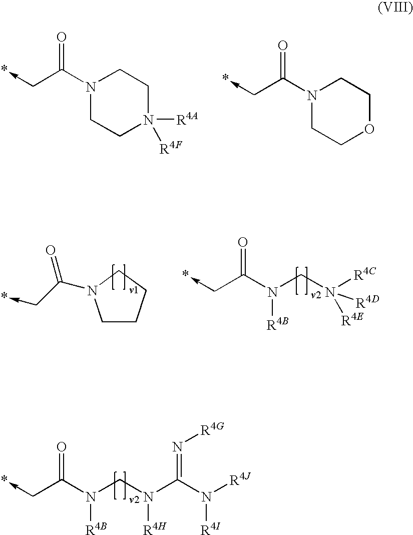

wherein
R^{4A}, R^{4B}, R^{4D}, R^{4E}, R^{4G}, R^{4H}, R^{4I} and R^{4J} are independently hydrogen or alkyl, or R^{4H} and R^{4I} may be joined together with the nitrogen atom to which they are attached to form a ring;
R^{4F} is a lone pair or R^{4F} is alkyl and the nitrogen atom to which it is attached is quaternary and carries a positive charge;
R^{4C} is a lone pair or R^{4C} is alkyl and the nitrogen atom to which it is attached is quaternary and carries a positive charge; or any two of R^{4C}, R^{4D} or R^{4E} may be joined together with the nitrogen atom to which they are attached to form a ring, optionally containing a further heteroatom selected from oxygen or nitrogen;
v1 is 1-3;
v2 is 1-6;

R⁵ is alkyl, which can be substituted with one to three identical or different radicals selected from the group consisting of halogen, hydroxy, alkoxy, alkenoxy, alkylthio, amino, hydroxycarbonyl, alkoxycarbonyl and the radical —O-(alkyl)-O-(alkyl); or R⁵ is amino;

R⁶ is halogen, nitro, cyano, alkyl, hydroxy or alkoxy, wherein alkyl and alkoxy can be further substituted with one to three identical or different radicals selected from the group consisting of halogen, hydroxy and alkoxy; and

Y¹, Y², Y³, Y⁴ and Y⁵ are independently each C or N, with the proviso that the ring in which they are comprised contains no more than 2 N atoms;

L is a linker group of Formula (III)

L^{a}-R⁷-L^{b}-W-L^{b}-R⁷-L^{a}- . . . 9 (III)

wherein

L^{a} is a bond or group —C(O)—;

L^{b} is a bond or group —C(O)—;

R⁷ is a bond or an alkylene or cycloalkylene group;

W is a bond or is selected from the following divalent radicals

—(O—R^{8A})ₘ₁—O—

—N(R^{9A})—(O—R^{8A})ₘ₁—R^{8A}—N(R^{9A})—

N(R^{9A})—R^{8B}—N(R^{9B})(R^{9C})—R^{8B}—N(R^{9A})—

N(R^{9A})—R^{8B}—N(R^{10B})C(═NR^{10A})(NR^{10C})—R^{8B}—N(R^{9A})—

—N(R^{9A})—R^{8B}—N(R^{9A})—

—N(R^{9A})—R^{8B}—N(R^{9A})—R^{8B}—N(R^{9A})—R^{8B}—N(R^{9A})—

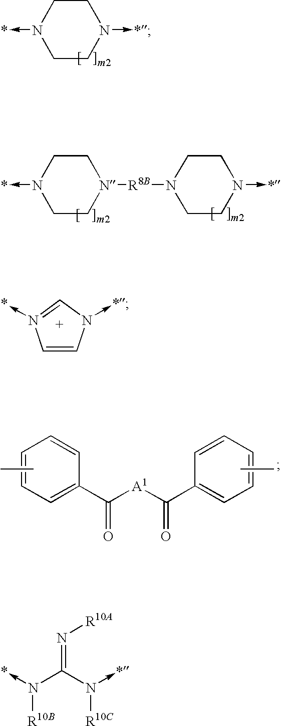

wherein

m1 is 14;

R^{8A} is an alkylene or cycloalkylene group;

R^{8B} is an alkylene or cycloalkylene group, or a group of Formula A²;

R^{9A} is hydrogen or alkyl;

one of R^{9B} or R^{9C} is a lone pair and the other is hydrogen or alkyl, or R^{9B} and R^{9C} are both alkyl, in which case the nitrogen to which they are attached is quaternary and carries a positive charge; or R^{9B} and R^{9C} may be joined together with the nitrogen to which they are attached to form a ring;

R^{10A} is hydrogen or alkyl;

R^{10B} and R^{10C} are independently hydrogen or alkyl, or alternatively R^{10B} and R^{10C} may be joined together to form a ring;

m2 is 1-3;

A¹ is selected from the groups —N(R^{9A})—R⁸—N(R^{9B})(R^{9C})—R⁸—N(R^{9A})—, —N(R^{9A})—R⁸—N(R^{10B})C(═NR^{10A}(NR^{10C})—R⁸—N(R^{9A})—;

A² is selected from one of the following groups;

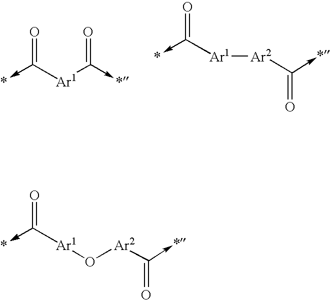

wherein Ar¹, Ar² are independently an aryl or heteroaryl group;

or a pharmaceutically acceptable salt, solvate or N-oxide thereof.

Compounds of the invention may be described as dimers, when there are two groups M. The linker L may however carry one or more further groups M.

It will be appreciated that any compound of the invention may be used in the form of a prodrug.

Compounds of the invention may be useful in the treatment or prevention of diseases in which HNE is implicated, for example chronic obstructive pulmonary disease (COPD), chronic bronchitis, lung fibrosis, pneumonia, acute respiratory distress syndrome (ARDS), pulmonary emphysema, smoking-induced emphysema, severe asthma, sarcoidosis, bronchiectasis, cystic fibrosis, inflammatory bowel disease; ulcerative colitis and Crohn's disease.

Another aspect of the invention is a pharmaceutical composition comprising a compound of the invention and a pharmaceutically acceptable carrier or excipient.

Another aspect of the invention is the use of a compound of the invention for the manufacture of a medicament for the treatment or prevention of a disease or condition in which HNE is implicated. Thus, compounds of the invention may be used in a method of therapy, for the treatment of a patient suffering from a condition or disease as defined above.

### DESCRIPTION OF PREFERRED EMBODIMENTS

“Alkylcarbonyl” means a —CO-alkyl group in which the alkyl group is as described herein. Exemplary acyl groups include —COCH₃ and —COCH(CH₃)₂.

“Acylamino” means a —NR-acyl group in which R and acyl are as described herein. Exemplary acylamino groups include —NHCOCH₃ and —N(CH₃)COCH₃.

“Alkenoxy” means an —O-alkenyl group in which alkenyl is as described below. Exemplary groups includes —O-allyl (—OCH₂CH═CH₂)

“Alkenoxycarbonyl” means a —COO-alkenyl group which alkenyl is as described below. Exemplary groups includes —C(O)O-allyl.

“Alkoxy” and “alkyloxy” means an —O-alkyl group in which alkyl is as described below. Exemplary alkoxy groups include methoxy (—OCH₃) and ethoxy (—OC₂H₅).

“Alkoxycarbonyl” means a —COO-alkyl group in which alkyl is as defined below. Exemplary alkoxycarbonyl groups include methoxycarbonyl and ethoxycarbonyl.

“Alkyl” or “lower alkyl”, as a group or part of a group, refers to a straight or branched chain saturated hydrocarbon group having from 1 to 12, preferably 1 to 6, carbon atoms, in the chain. Exemplary alkyl groups include methyl, ethyl, 1-propyl and 2-propyl.

“Alkenyl” as a group or part of a group refers to a straight or branched chain hydrocarbon group having from 1 to 12, preferably 1 to 6, carbon atoms and one carbon-carbon double bond in the chain. Exemplary alkenyl groups include ethenyl, 1-propenyl, and 2-propenyl.

“Alkylamino” means a —NH-alkyl group in which alkyl is as defined above. Exemplary alkylamino groups include methylamino and ethylamino.

“Alkylene means an -alkyl- group in which alkyl is as defined previously. Exemplary alkylene groups include —CH₂—, —(CH₂)₂— and —C(CH₃)HCH₂—.

“Alkenylene” means an -alkenyl- group in which alkenyl is as defined previously. Exemplary alkenylene groups include —CH═CH—, —CH═CHCH₂—, and —CH₂CH═CH—.

“Alkylthio” means a —S-alkyl group in which alkyl is as defined above. Exemplary alkylthio groups include methylthio and ethylthio.

“Amino” means a —NR¹R² group where R¹ and R² may be independently a hydrogen atom, alkyl, aryl, arylalkyl, alkenyl, alkynyl, heteroaryl or heterocycloalkyl group. (i.e. The amino group may be primary, secondary or tertiary). Exemplary amino groups include —NH₂, NHCH₃, —NHPh, —N(CH₃)₂, etc.

“Aminocarbonyl” means a —CO—NRR group in which R is as herein described. Exemplary aminocarbonyl groups include —CONH₂, —CONHCH₃ and —CONH-phenyl.

“Aminoalkyl” means an alkyl-NH₂ group in which alkyl is as previously described. Exemplary aminoalkyl groups include —CH₂NH₂.

“Ammonium” means a quarternary nitrogen group —N⁺R¹R²R³ where R¹, R² and R³ are alkyl, aryl, alkenyl, arylalkyl, heteroaryl, heterocycloalkyl, and the nitrogen atom carries a formal positive charge.

“Aryl” as a group or part of a group denotes an optionally substituted monocyclic or multicyclic aromatic carbocyclic moiety of from 6 to 14 carbon atoms, preferably from 6 to 10 carbon atoms, such as phenyl or naphthyl. The aryl group may be substituted by one or more substituent groups.

“Arylalkyl” means an aryl-alkyl- group in which the aryl and alkyl moieties are as previously described. Exemplary arylalkyl groups include benzyl, phenethyl and naphthlenemethyl.

“Arylalkyloxy” means an aryl-alkyloxy- group in which the aryl and alkyloxy moieties are as previously described. Preferred arylalkyloxy groups contain a C₁₋₄ alkyl moiety. Exemplary arylalkyl groups include benzyloxy.

“Arylcarbonyl” means an aromatic ring joined to a carbonyl group (C═O). Exemplary groups include benzoyl (—C(O)Ph).

“Aryloxy” means an —O-aryl group in which aryl is described above. Exemplary aryloxy groups include phenoxy.

“Cyclic amine” means an optionally substituted 3 to 8 membered monocyclic cycloalkyl ring system where one of the ring carbon atoms is replaced by nitrogen, and which may optionally contain an additional heteroatom selected from O, S or NR (where R is as described herein). Exemplary cyclic amines include pyrrolidine, piperidine, morpholine, piperazine and N-methylpiperazine. The cyclic amine group may be substituted by one or more substituent groups.

“Cycloalkyl” means an optionally substituted saturated monocyclic or bicyclic ring system of from 3 to 12 carbon atoms, preferably from 3 to 8 carbon atoms, and more preferably from 3 to 6 carbon atoms. Exemplary monocyclic cycloalkyl rings include cyclopropyl, cyclopentyl, cyclohexyl and cycloheptyl. The cycloalkyl group may be substituted by one or more substituent groups.

“Cyloalkylene” means an optionally substituted saturated monocyclic or bicyclic ring system of from 3 to 12 carbon atoms, preferably from 3 to 8 carbon atoms, and more preferably from 3 to 6 carbon atoms, as a bivalent radical. Exemplary cycloalkylene groups include cyclohexane-1,4-diyl.

“Cycloalkylalkyl” means a cycloalkyl-alkyl- group in which the cycloalkyl and alkyl moieties are as previously described. Exemplary monocyclic cycloalkylalkyl groups include cyclopropylmethyl, cyclopentylmethyl, cyclohexylmethyl and cycloheptylmethyl.

“Dendrimer” means a multifunctional core group with a branching group attached to each functional site. Each branching site can be attached to another branching molecule and this process may be repeated multiple times.

“Halo” or “halogen” means fluoro, chloro, bromo, or iodo.

“Haloalkoxy” means an —O-alkyl group in which the alkyl is substituted by one or more halogen atoms. Exemplary haloalkyl groups include trifluoromethoxy and difluoromethoxy.

“Haloalkyl” means an alkyl group which is substituted by one or more halo atoms. Exemplary haloalkyl groups include trifluoromethyl.

“Heteroaryl” as a group or part of a group denotes an optionally substituted aromatic monocyclic or multicyclic organic moiety of from 5 to 14 ring atoms, preferably from 5 to 10 ring atoms, in which one or more of the ring atoms is/are element(s) other than carbon, for example nitrogen, oxygen or sulfur. Examples of such groups include benzimidazolyl, benzoxazolyl, benzothiazolyl, benzofuranyl, benzothienyl, furyl, imidazolyl, indolyl, indolizinyl, isoxazolyl, isoquinolinyl, isothiazolyl, oxazolyl, oxadiazolyl, pyrazinyl, pyridazinyl, pyrazolyl, pyridyl, pyrimidinyl, pyrrolyl, quinazolinyl, quinolinyl, tetrazolyl, 1,3,4-thiadiazolyl, thiazolyl, thienyl and triazolyl groups. The heteroaryl group may be substituted by one or more substituent groups. The heteroaryl group may be attached to the remainder of the compound of the invention by any available carbon or nitrogen atom.

“Heteroarylcarbonyl” means a heteroaryl group attached to a carbonyl group —C(O)—. Exemplary groups are pyridine-2-carbonyl, thiophene-2-carbonyl.

“Heteroaryloxy” means a heteroaryloxy- group in which the heteroaryl is as previously described. Exemplary heteroaryloxy groups include pyridyloxy.

“Heterocycloalkyl” means: (i) an optionally substituted cycloalkyl group of from 4 to 8 ring members which contains one or more heteroatoms selected from O, S or NR; (ii) a cycloalkyl group of from 4 to 8 ring members which contains CONR and CONRCO (examples of such groups include succinimidyl and 2-oxopyrrolidinyl). The heterocycloalkyl group may be substituted by one or more substituent groups. The heterocycloalkyl group may be attached to the remainder of the compound by any available carbon or nitrogen atom.

“Heterocycloalkylalkyl” means a heterocycloalkyl-alkyl- group in which the heterocycloalkyl and alkyl moieties are as previously described.

“Hydroxycarbonyl” means a group —COOH.

“Pharmaceutically acceptable salt” means a physiologically or toxicologically tolerable salt and include, when appropriate, pharmaceutically acceptable base addition salts and pharmaceutically acceptable acid addition salts. For example (i) where a compound of the invention contains one or more acidic groups, for example carboxy groups, pharmaceutically acceptable base addition salts that may be formed include sodium, potassium, calcium, magnesium, and ammonium salts, or salts with organic amines, such as, diethylamine, N-methyl-glucamine, diethanolamine or amino acids (e.g. lysine) and the like; (ii) where a compound of the invention contains a basic group, such as an amino group, pharmaceutically acceptable acid addition salts that may be formed include hydrochlorides, hydrobromides, phosphates, acetates, citrates, lactates, tartrates, malonates, methanesulphonates and the like. “Pharmaceutically acceptable salt” also means quaternary ammonium salts. In this case, the acceptable salts may be chlorides, bromides, iodides, mesylates, tosylates, succinates and the like.

It will be understood that, as used herein, references to the compounds of the invention are meant to also include the pharmaceutically acceptable salts.

“Prodrug” refers to a compound which is convertible in vivo by metabolic means (e.g. by hydrolysis, reduction or oxidation) to a compound of the invention. For example an ester prodrug of a compound of the invention containing a hydroxy group may be convertible by hydrolysis in vivo to the parent molecule. Suitable esters of compounds of the invention containing a hydroxy group, are for example acetates, citrates, lactates, tartrates, malonates, oxalates, salicylates, propionates, succinates, fumarates, maleates, methylene-bis-p-hydroxynaphthoates, gentisates, isethionates, di-p-toluoyltartrates, methanesulfonates, ethanesulfonates, benzenesulfonates, p-toluenesulfonates, naphthalene bis sulfonates, cyclohexylsulfamates and quinates. As another example an ester prodrug of a compound of the invention containing a carboxy group may be convertible by hydrolysis in vivo to the parent molecule. Examples of ester prodrugs are those described by F. J. Leinweber, Drug Metab. Res., 1987, 18, 379.

It will be understood that, as used in herein, references to the compounds of the invention are meant to also include the prodrug forms.

“Saturated” pertains to compounds and/or groups which do not have any carbon-carbon double bonds or carbon-carbon triple bonds.

The cyclic groups referred to above, namely, aryl, heteroaryl, cycloalkyl, and heterocycloalkyl, may be substituted by one or more substituent groups. Suitable optional substituent groups include acyl (e.g. —COCH₃), alkoxy (e.g., —OCH₃), alkoxycarbonyl (e.g. —COOCH₃), alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoacyl (e.g. —CON(CH₃)₂), aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), —OH, —CHO, —NO₂, aryl (optionally substituted with alkoxy, haloalkoxy, halogen, alkyl or haloalkyl), heteroaryl (optionally substituted with alkoxy, haloalkoxy, halogen, alkyl or haloalkyl), heterocycloalkyl, aminoacyl (e.g. —CONH₂, —CONHCH₃), aminosulfonyl (e.g. —SO₂NH₂, —SO₂NHCH₃), acylamino (e.g. —NHCOCH₃), sulfonylamino (e.g. —NHSO₂CH₃), heteroarylalkyl, cyclic amine (e.g. morpholine), aryloxy, heteroaryloxy, arylalkyloxy (e.g. benzyloxy) and heteroarylalkyloxy.

Alkylene or alkenylene groups may be optionally substituted. Suitable optional substituent groups include alkoxy (e.g., —OCH₃), alkylamino (e.g. —NHCH₃), alkylsulfinyl (e.g. —SOCH₃), alkylsulfonyl (e.g. —SO₂CH₃), alkylthio (e.g. —SCH₃), —NH₂, aminoalkyl (e.g. —CH₂NH₂), arylalkyl (e.g. —CH₂Ph or —CH₂—CH₂-Ph), cyano, dialkylamino (e.g. —N(CH₃)₂), halo, haloalkoxy (e.g. —OCF₃ or —OCHF₂), haloalkyl (e.g. —CF₃), alkyl (e.g. —CH₃ or —CH₂CH₃), —OH, —CHO, and —NO₂.

Compounds of the invention may exist in one or more geometrical, optical, enantiomeric, diastereomeric and tautomeric forms, including but not limited to cis and trans-forms, E- and Z-forms, R-, S- and meso-forms, keto-, and enol-forms. Unless otherwise stated a reference to a particular compound includes all such isomeric forms, including racemic and other mixtures thereof. Where appropriate such isomers can be separated from their mixtures by the application or adaptation of known methods (e.g. chromatographic techniques and recrystallisation techniques). Where appropriate such isomers may be prepared by the application of adaptation of known methods (e.g. asymmetric synthesis).

G may be a group of any of formulae (V) to (VII) or a dendrimer. Examples of groups of formulae (V) to (VII) include, but are not limited to phenoxyphenyl, biphenyl, bipyridyl, ethylenediamino, propylenediamino and the like. It is to be understood that the number of possible attachment points is dictated by the valency of the groups present, so that for example, biphenyl can contain up to 10 possible attachments (5 on each phenyl ring), and ethylenediamine can possess up to 4 possible attachments (2 on each terminal amine). An example of a dendrimer suitable for use in the invention is:

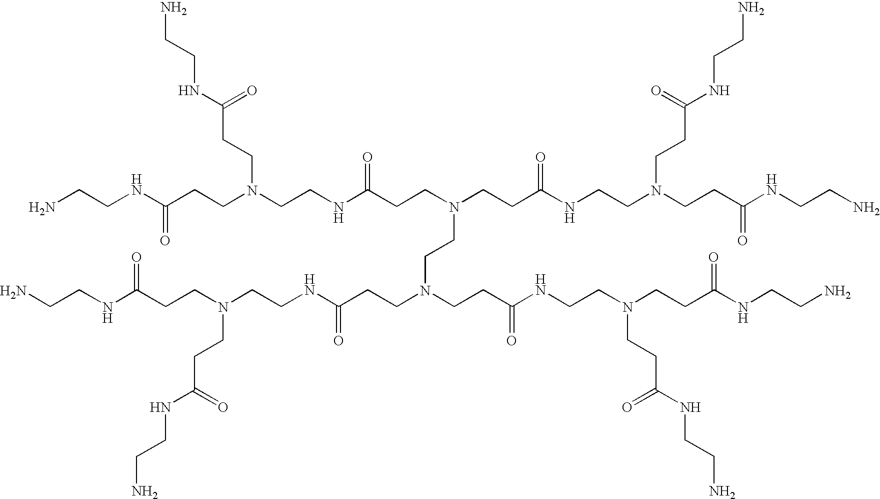

Certain compounds and combinations of substituents are preferred. Certain preferences are given in the subclaims.

In a preferred embodiment, each M is the same or different and is a group of formula (II) as defined herein. In formula (II), the arrow denotes the point of attachment of M to the linker L. Preferably, L is a group of Formula (III) as defined herein.

In a preferred embodiment, compounds are of Formula (I).

In a preferred embodiment, A is a phenyl ring.

In one embodiment, R¹ is a cyano group and R² is a hydrogen atom.

In a preferred embodiment, D is an oxygen atom.

In another preferred embodiment, the groups M have the stereochemistry shown below;

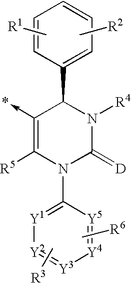

In one preferred embodiment R⁶ is a haloalkyl group.

In one preferred embodiment, Y¹-Y⁵ are carbon atoms.

In a preferred embodiment, L^{a} is a group C(O).

In one preferred embodiment R⁷ and L^{b} are a bond.

In a preferred embodiment, W is a radical

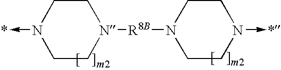

In a further preferred embodiment, W is the radical —N(R^{9A})—R^{8B}—N(R^{9B})(R^{9C})—R^{8B}—N(R^{9A})—.

In another preferred embodiment, W is the radical —N(R^{9A})—R^{8B}—N(R^{10B})C(═NR^{10A})(NR^{10C})—R^{8B}—N(R^{9A})—.

In another preferred embodiment, W is the radical —N(R^{9A})—R^{8B}—N(R^{9A})—R^{8B}—N(R^{9A})—R^{8B}—N(R^{9A})—

In yet another preferred embodiment R⁵ is an alkyl group.

In yet another preferred embodiment R⁵ is a methyl group.

In one embodiment R⁴ is a hydrogen atom

In a further embodiment R⁴ is a group of Formula (VIII)

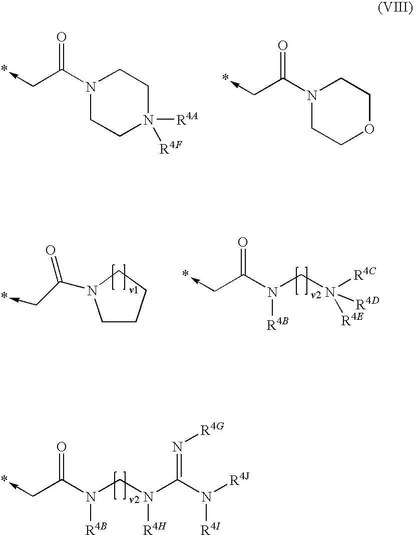

Preferred compounds of the invention include those of the Examples, e.g. of Examples 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 32, 34, 36, 37 and 38.

The therapeutic utility of the present compounds is pertinent to any disease that is known to be at least partially mediated by the action of human neutrophil elastase. For example, the present compounds may be beneficial in the treatment of chronic obstructive pulmonary disease (COPD), cystic fibrosis (CF), acute respiratory distress syndrome (ARDS), pulmonary emphysema, pneumonia and lung fibrosis.

The present invention is also concerned with pharmaceutical formulations comprising, as an active ingredient, a compound of the invention. Other compounds may be combined with compounds of this invention for the prevention and treatment of inflammatory diseases of the lung. Thus the present invention is also concerned with pharmaceutical compositions for preventing and treating inflammatory diseases of the lung comprising a therapeutically effective amount of a compound of the invention and one or more other therapeutic agents.

Suitable therapeutic agents for a combination therapy with compounds of the invention include: (1) a corticosteroid, for example fluticasone, ciclesonide or budesonide; (2) a β2-adrenoreceptor agonist, for example salmeterol, indacaterol or formeterol; (3) a leukotriene modulator, for example montelukast or praniukast; (4) muscarinic-3 (M3) receptor antagonists such as botropium bromide; (5) bronchodilators that possess both M3 receptor antagonism and β2-adrenoreceptor agonism in a single molecule (6) phosphodiesterase-IV (PDE-IV) inhibitors, for example roflumilast or cilomilast; (7) an antitussive agent, such as codeine or dextramorphan; (8) a non-steroidal anti-inflammatory agent (NSAID), for example ibuprofen or ketoprofen; (9) kinase inhibitors such as p38 MAP kinase inhibitors, IKK2 inhibitors and (10) receptor antagonists for cytokines and chemokines for example IL-8, MCP-1, TNFα and IL-1β

The weight ratio of the first and second active ingredients may be varied and will depend upon the effective dose of each ingredient. Generally, an effective dose of each will be used.

The magnitude of prophylactic or therapeutic dose of a compound of the invention will, of course, vary with the nature of the severity of the condition to be treated and with the particular compound and its route of administration. It will also vary according to the age, weight and response of the individual patient. In general, the daily dose range will lie within the range of from about 0.001 mg to about 100 mg per kg body weight of a mammal, preferably 0.01 mg to about 50 mg per kg, and most preferably 0.1 to 10 mg per kg, in single or divided doses. On the other hand, it may be necessary to use dosages outside these limits in some cases.

Another aspect of the present invention provides pharmaceutical compositions which comprise a compound of the invention and a pharmaceutically acceptable carrier. The term “composition”, as in pharmaceutical composition, is intended to encompass a product comprising the active ingredient(s), and the inert ingredient(s) (pharmaceutically acceptable excipients) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the invention, additional active ingredient(s), and pharmaceutically acceptable excipients.

The pharmaceutical compositions of the present invention comprise a compound of the invention as an active ingredient or a pharmaceutically acceptable salt thereof, and may also contain a pharmaceutically acceptable carrier and optionally other therapeutic ingredients. The term “pharmaceutically acceptable salts” refers to salts prepared from pharmaceutically acceptable non-toxic bases or acids including inorganic bases or acids and organic bases or acids.

Any suitable route of administration may be employed for providing a mammal, especially a human, with an effective dosage of a compound of the present invention. In therapeutic use, the active compound may be administered by any convenient, suitable or effective route. Suitable routes of administration are known to those skilled in the art, and include oral, intravenous, rectal, parenteral, topical, ocular, nasal, buccal and pulmonary. Delivery by inhalation is preferred.

Compositions suitable for administration by inhalation are known, and may include carriers and/or diluents that are known for use in such compositions. The composition may contain 0.01-99% by weight of active compound. Preferably, a unit dose comprises the active compound in an amount of 1 μg to 10 mg.

The most suitable dosage level may be determined by any suitable method known to one skilled in the art. It will be understood, however, that the specific amount for any particular patient will depend upon a variety of factors, including the activity of the specific compound that is used, the age, body weight, diet, general health and sex of the patient, time of administration, the route of administration, the rate of excretion, the use of any other drugs, and the severity of the disease undergoing treatment.

For delivery by inhalation, the active compound is preferably in the form of microparticles. They may be prepared by a variety of techniques, including spray-drying, freeze-drying and micronisation.

By way of example, a composition of the invention may be prepared as a suspension for delivery from a nebuliser or as an aerosol in a liquid propellant, for example for use in a pressurised metered dose inhaler (PMDI). Propellants suitable for, use in a PMDI are known to the skilled person, and include CFC-12, HFA-134a, HFA-227, HCFC-22 (CCl2F2) and HFA-152 (CH4F2 and isobutane).

In a preferred embodiment of the invention, a composition of the invention is in dry powder form, for delivery using a dry powder inhaler (DPI). Many types of DPI are known.

Microparticles for delivery by administration may be formulated with excipients that aid delivery and release. For example, in a dry powder formulation, microparticles may be formulated with large carrier particles that aid flow from the DPI into the lung. Suitable carrier particles are known, and include lactose particles; they may have a mass median aerodynamic diameter of greater than 90 μm.

In the case of an aerosol-based formulation, a preferred composition is:

Compound of the invention 24 mg/canister

Lecithin, NF Liq. Conc. 1.2 mg/canister

Trichlorofluoromethane, NF 4.025 g/canister

Dichlorodifluoromethane, NF 12.15 g canister.

Compounds of the invention may be used in combination with other drugs that are used in the treatment/prevention/suppression or amelioration of the diseases or conditions for which present compounds are useful. Such other drugs may be administered, by a route and in an amount commonly used therefore, contemporaneously or sequentially with a compound of the invention. When a compound of the invention is used contemporaneously with one or more other drugs, a pharmaceutical composition containing such other drugs in addition to the compound of the invention is preferred. Accordingly, the pharmaceutical compositions of the present invention include those that also contain one or more other active ingredients, in addition to a compound of the invention.

The agents of the invention may be administered in inhaled form. Aerosol generation can be carried out using, for example, pressure-driven jet atomizers or ultrasonic atomizers, preferably using propellant-driven metered aerosols or propellant-free administration of micronized active compounds from, for example, inhalation capsules or other “dry powder” delivery systems.

The active compounds may be dosed as described depending on the inhaler system used. In addition to the active compounds, the administration forms may additionally contain excipients, such as, for example, propellants (e.g. Frigen in the case of metered aerosols), surface-active substances, emulsifiers, stabilizers, preservatives, flavorings, fillers (e.g. lactose in the case of powder inhalers) or, if appropriate, further active compounds.

For the purposes of inhalation, a large number of systems are available with which aerosols of optimum particle size can be generated and administered, using an inhalation technique which is appropriate for the patient. In addition to the use of adaptors (spacers, expanders) and pear-shaped containers (e.g. Nebulator®, Volumatic®), and automatic devices emitting a puffer spray (Autohaler®), for metered aerosols, in particular in the case of powder inhalers, a number of technical solutions are available (e.g. Diskhaler®, Rotadisk®, Turbohaler® or the inhalers for example as described EP-A-0505321).

The compounds of the invention of the present invention can be prepared according to the procedures of the following schemes and examples, using appropriate materials, and are further exemplified by the following specific examples. Moreover, by utilising the procedures described with the disclosure contained herein, one of ordinary skill in the art can readily prepare additional compounds of the present invention claimed herein. The compounds illustrated in the examples are not, however, to be construed as forming the only genus that is considered as the invention. The examples further illustrate details for the preparation of the compounds of the present invention. Those skilled in the art will readily understand that known variations of the conditions and processes of the following preparative procedures can be used to prepare these compounds.

The compounds of the invention may be isolated in the form of their pharmaceutically acceptable salts, such as those described previously herein above. The free acid form corresponding to isolated salts can be generated by neutralisation with a suitable acid such as acetic acid and hydrochloric acid and extraction of the liberated free acid into an organic solvent followed by evaporation. The free acid form isolated in this manner can be further converted into another pharmaceutically acceptable salt by dissolution in an organic solvent followed by addition of the appropriate base and subsequent evaporation, precipitation, or crystallisation.

It may be necessary to protect reactive functional groups (e.g. hydroxy, amino, thio or carboxy) in intermediates used in the preparation of compounds of the invention to avoid their unwanted participation in a reaction leading to the formation of the compounds. Conventional protecting groups, for example those described by T. W. Greene and P. G. M. Wuts in “Protective groups in organic chemistry” John Wiley and Sons, 1999, may be used.

The following reaction Schemes illustrate how compounds of the invention, in particular the Example compounds, may be prepared. It will be understood that the processes detailed below are solely for the purpose of illustrating the invention and should not be construed as limiting. A process utilising similar or analogous reagents and/or conditions known to one skilled in the art may also be used to obtain a compound of the invention.

The following reaction schemes illustrate how compounds of the invention, in particular the Example compounds, may be prepared. It will be understood that the processes detailed below are solely for the purpose of illustrating the invention and should not be construed as limiting. A process utilising similar or analogous reagents and/or conditions known to one skilled in the art may also be used to obtain a compound of the invention.

The monomers may be joined together by a range of standard chemistries, for example, reaction with a suitable bifunctional linker molecule in the presence of a suitable reagent and optional solvent, Scheme 1 (Route A). An alternative methodology involves attachment of a spacer group incorporating a second functional group, which subsequently allows attachment of a bidentate linker molecule, Scheme 1 (Route B). The linker part of the dimer may be modified after dimerisation.

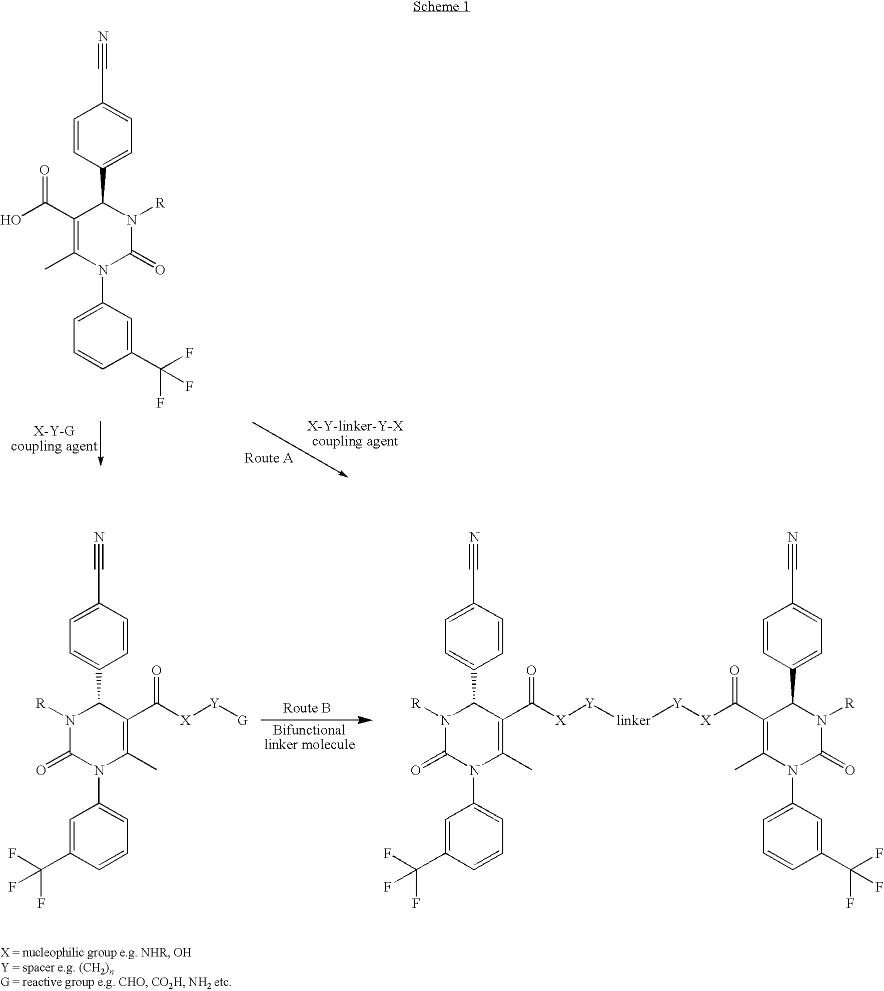

More specifically to the example compounds, reaction of the monomer with a suitable diamine or diol can be effected in the presence of a base and coupling reagent, for example HATU, and optional solvent. Reaction of the monomer with a protected aminoaldehyde (protected as an acetal), in the presence of a suitable base and coupling reagent, and optional solvent, followed by deprotection leads to an intermediate that can be reacted with a suitable bidentate species, such as a diamine, to generate compounds of the invention, Scheme 2.

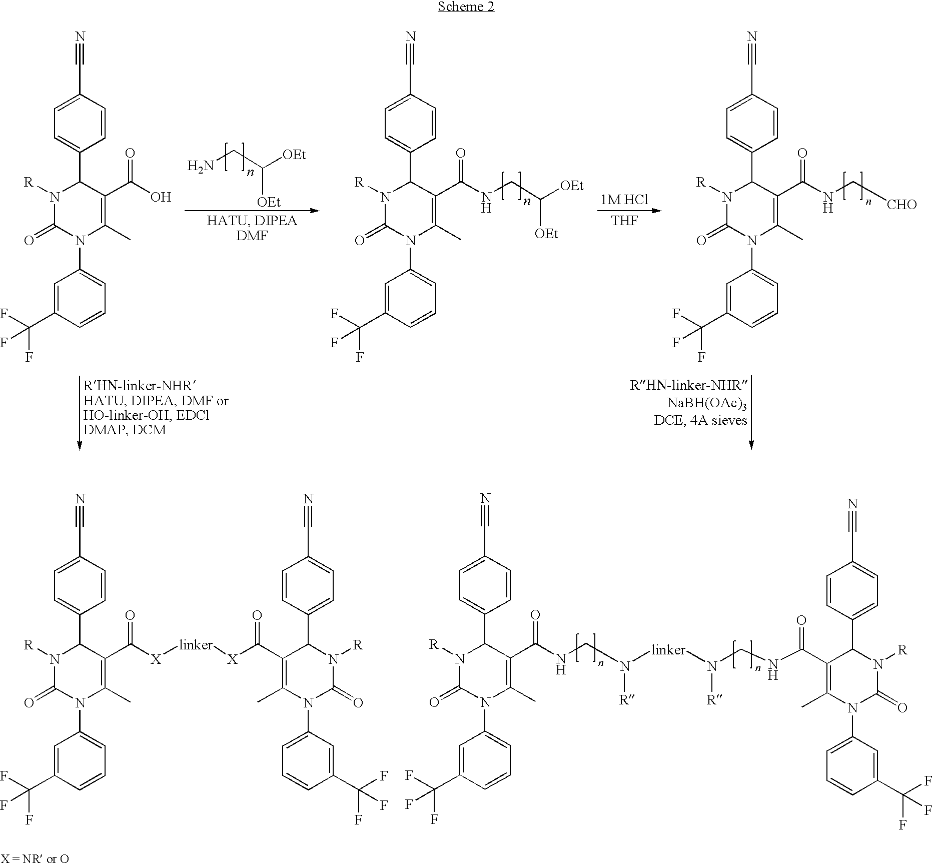

The monomers of Formula (II) may be prepared as racemates according to methods described in WO2004024700, WO2004024701, GB2392910, WO200508263 and WO2005082864. The monomers may be separated by chiral HPLC into their enantiomers. Alternatively the monomer (as a carboxylic acid) may be resolved by formation of diastereomeric salts with a suitable chiral base, such as norephedrine, followed by fractional recrystallisation, Scheme 3.

N-3 may be functionalised via alkylation with a suitably activated alkane in the presence of a suitable base, for example a metal hydride, and optional solvent. Suitable leaving groups include halogen and sulfonate. Similarly, N-3 of the monomer may be acylated with an acid halide under similar conditions. This group (R in Schemes 1-4) may be modified further in subsequent steps. Prior protection of the carboxylic acid by, for example, conversion to the corresponding allyl ester, is required to prevent unwanted side-reactions.

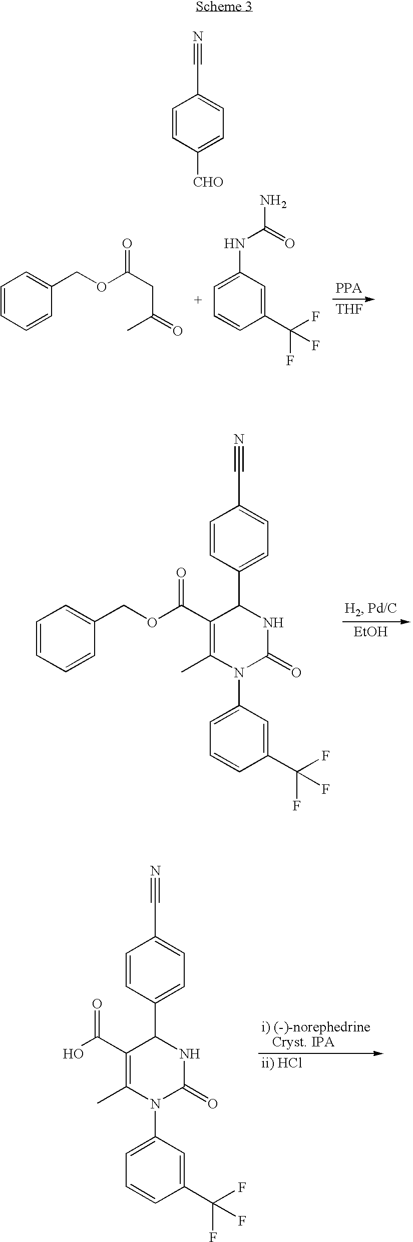

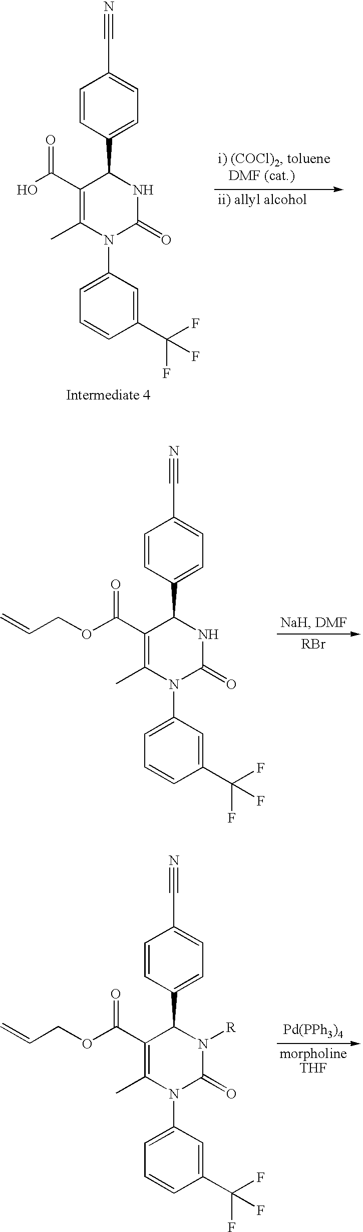

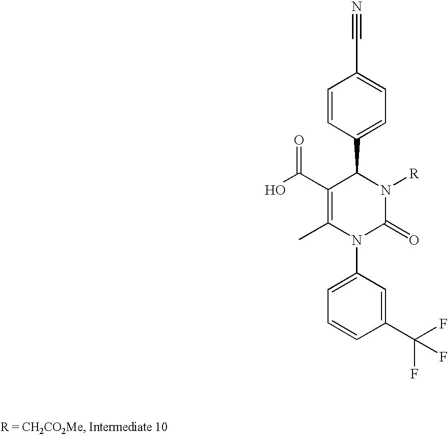

Compounds of Formula (II) containing a cyclic guanidine moiety in the linker group may be prepared according to the method outlined in Scheme 4.

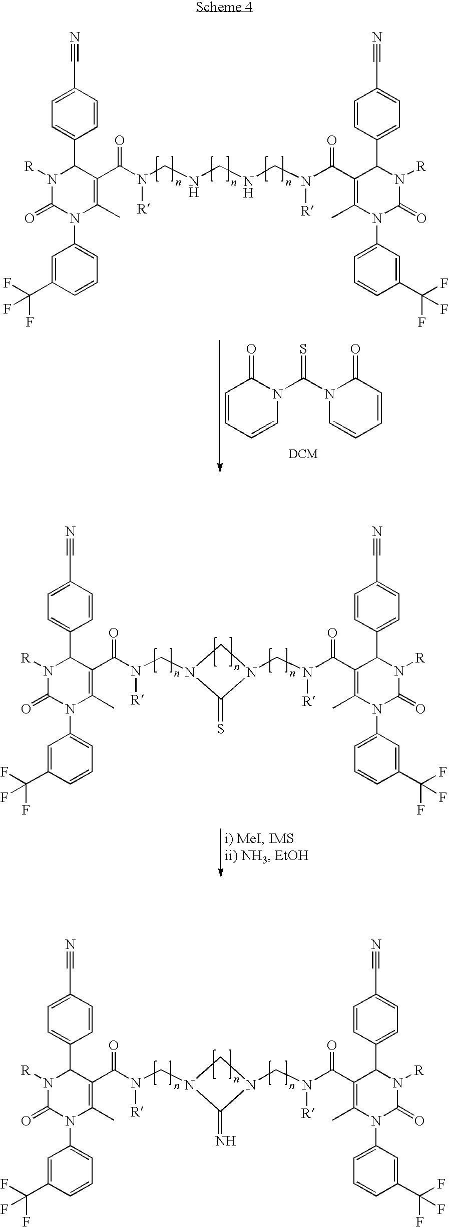

Compounds that are quaternary ammonium salts can be generated by reaction of the parent amine with a suitably activated alkane. Suitable leaving groups include halogen and sulfonate.

The following Examples illustrate the invention.

### General Experimental Details:

Where products were purified using an Isolute SPE Si II cartridge, ‘Isolute SPE Si cartridge’ refers to a pre-packed polypropylene column containing unbonded activated silica with irregular particles with average size of 50 μm and nominal 60 Å porosity. Where an Isolute SCX-2 cartridge is used, ‘Isolute SCX-2 cartridge’ refers to a pre-packed polypropylene column containing a non end-capped propylsulphonic acid functionalised silica strong cation exchange sorbent. All solvents and commercial reagents were used as received. After HPLC purification, fractions containing product were combined and freeze-dried to give the product as a white or off-white solid. In some cases, where the compound contained a basic centre, the product was obtained as the formate salt.

### Preparative HPLC Conditions:

### HPLC System 1:

C18-reverse-phase column (100×22.5 mm i.d Genesis column with 7 μm particle size), eluting with a gradient of A: water+0.1% formic acid; B: acetonitrile+0.1% formic acid at a flow rate of 5 ml/min and gradient of 1%/min increasing in B. UV detection at 230 nm.

### HPLC System 2:

Phenyl hexyl column (250×21.20 mm Luna column with 5 μm particle size), eluting with a gradient of A: water+0.1% TFA; B: acetonitrile+0.1% TFA at a flow rate of 5 ml/min with UV detection at 254 nm.

### HPLC System 3:

Amylose tris(3,5-dimethylphenylcarbamate) (250×20 mm CHIRALPAK IA column with 5 μm particle size), eluting with an isocratic mixture of ethanol (15%) in n-heptane+0.1% TFA at a flow rate of 10 ml/min with UV detection at 254 nm.

The Liquid Chromatography Mass Spectroscopy (LC/MS) systems used:

### LC-MS Method 1:

Micromass Platform LCT with a C18-reverse-phase column (100×3.0 mm Higgins Clipeus with 5 μm particle size), elution with A: water+0.1% formic acid; B: acetonitrile+0.1% formic acid. Gradient:

| Gradient - Time | flow ml/min | % A | % B |
|---|---|---|---|
| 0.00 | 1.0 | 95 | 5 |
| 1.00 | 1.0 | 95 | 5 |
| 15.00 | 1.0 | 5 | 95 |
| 20.00 | 1.0 | 5 | 95 |
| 22.00 | 1.0 | 95 | 5 |
| 25.00 | 1.0 | 95 | 5 |

Detection—MS, ELS, UV (100 μl split to MS with in-line UV detector)
MS ionisation method—Electrospray (positive ion)

### LC-MS Method 2:

Micromass Platform LCT with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow ml/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (100 μl split to MS with in-line UV detector)
MS ionisation method—Electrospray (positive and negative ion)

### LC-MS Method 3:

Waters Micromass ZQ with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow ml/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (100 μl split to MS with in-line UV detector)
MS ionisation method—Electrospray (positive and negative ion)

### LC-MS Method 4:

Waters Micromass ZMD with a C18-reverse-phase column (30×4.6 mm Phenomenex Luna 3 μm particle size), elution with A: water+0.1% formic acid; B: acetonitrile+0.1% formic acid. Gradient:

| Gradient - Time | flow ml/min | % A | % B |
|---|---|---|---|
| 0.00 | 2.0 | 95 | 5 |
| 0.50 | 2.0 | 95 | 5 |
| 4.50 | 2.0 | 5 | 95 |
| 5.50 | 2.0 | 5 | 95 |
| 6.00 | 2.0 | 95 | 5 |

Detection—MS, ELS, UV (100 μl split to MS with in-line UV detector)
MS ionisation method—Electrospray (positive and negative ion)

Abbreviations used in the experimental section:

DCM=dichloromethane
DCE=1,1-dichloroethane
DIPEA=di-isopropylethylamine
EDCI=1-(3-dimethylaminopropyl)-3-ethylcarbodiimide hydrochloride
DMAP=dimethylaminopyridine
RT=room temperature
HATU=O-(7-Azabenzotriazol-1-yl)-N,N,N′,N′-tetramethyluroniumhexafluorophosphate.
TFA=trifluoroacetic acid
Rt=retention time
IMS=industrial methylated spirits
THF=tetrahydrofuran

### DMF=N,N-dimethylformamide

IPA=isopropyl alcohol
SPE=solid phase extraction
SCX=strong cation exchange

### INTERMEDIATE 1

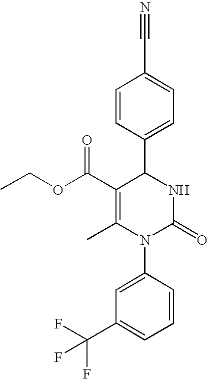

Intermediate 1 was prepared according to WO2004/024700.

### INTERMEDIATE 2

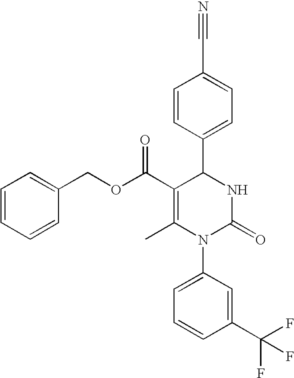

Polyphosphoric acid (350 g) was suspended in THF (1.9 l) and stirred, mechanically, whilst 3-(trifluoromethyl)phenylurea (129 g), 4-cyanobenzaldehyde (100 g) and benzyl acetoacetate (121.3 g) were added. The resulting mixture was heated at reflux overnight. The bulk of the solvent was evaporated under reduced pressure and the residue partitioned between water and EtOAc. The organic phase was washed with water, aqueous K₂CO₃ solution, water, brine, dried (Na₂SO₄) and evaporated. The residue was triturated with diethyl ether to give Intermediate 2 as a yellow solid.

Yield: 216.6 g (70%)

¹H-NMR (400 MHz, DMSO-d₆): δ=8.38 (1H, d), 7.84 (2H, m), 7.80-7.55 (6H m), 7.30 (3H, m), 7.15 (2H, m), 5.13 (1H, d), 5.06 (1H, d), 5.39 (1H, d), 2.08 (3H, s).

### INTERMEDIATE 3

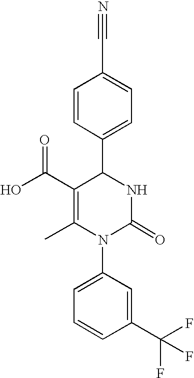

Intermediate 2 (215 g, 0.54 mol) was suspended in EtOAc (1.5 l) and IMS (500 ml) was added. The mixture was warmed slightly by immersion in a warm water bath (ca. 40° C.) until homogeneous and then treated, under nitrogen, with Pd(OH)₂ on carbon (20 g, 20% w/w, 50% water wet). The flask was sealed and evacuated and then the mixture was stirred under an atmosphere of hydrogen whilst keeping warm by replenishment of the warm water bath. After ca. 4-6 h, the flask was evacuated and the mixture filtered through ‘hyflo’. The filtrate was evaporated under vacuum and the residue triturated with diethyl ether to furnish Intermediate 3 as a solid which was used directly in the next step.

### INTERMEDIATE 4

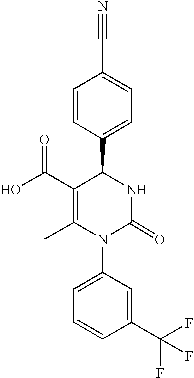

Intermediate 3 (156.2 g) was suspended in IPA (1500 ml) and treated with L-(−)-norephedrine. The mixture became homogeneous and then began to deposit a solid. After stirring for ca. 6 h the solid was filtered off and washed with IPA and sucked as dry as possible on the filter. The cake was removed and dissolved in a minimum amount of hot IPA (ca. 2.5 l) and allowed to cool, with stirring, overnight. The mixture was filtered and washed with IPA and dried to give a white solid. This was partitioned between EtOAc and 2M HCl until all solid had dissolved. The organic layer was washed with brine, dried (Na₂SO₄) and evaporated to give Intermediate 4 as a colourless foam (61.7 g).

Yield: 61.7 g (29%)

¹H-NMR (400 MHz, DMSO-d₆): δ=12.48 (1H, br s), 8.32 (1H, d), 7.88 (2H, m), 7.79-7.54 (6H, m), 5.36 (1H, d), 4.03 (2H, q), 2.05 (3H, s), 1.18 (3H, t).

### INTERMEDIATES 4 AND 5

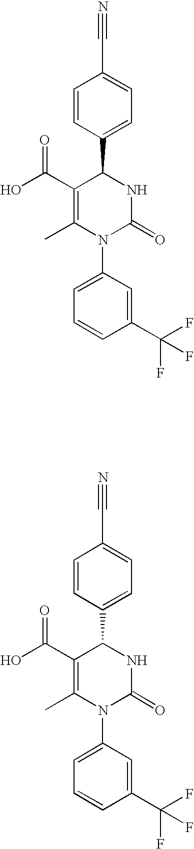

Intermediate 3 was separated into its enantiomers using HPLC (System 3). The first enantiomer to elute was Intermediate 5.

### INTERMEDIATE 6

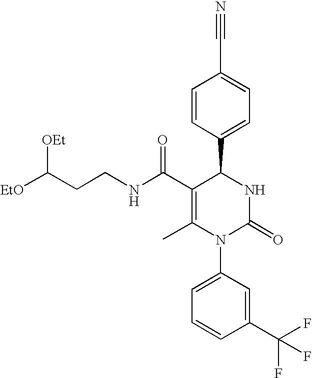

To a solution of Intermediate 4 (1.40 g, 3.49 mmol), 1-amino-2,3-diethoxypropane (513 mg, 3.49 mmol), and DIPEA (2.25 g, 17.45 mmol) in DMF (50 ml) was added HATU (1.592 g, 4.19 mmol). The solution was allowed to stand at RT for 2 h and the DMF was evaporated. The residue was partitioned between EtOAc (150 ml) and sat. aqueous NaHCO₃ (200 ml). The organic layer was separated and the aqueous was extracted further with EtOAc (2×150 ml). The combined extracts were washed with water (200 ml), brine (100 ml), dried (Na₂SO₄) and evaporated. The crude product was purified on an Isolute SPE Si II cartridge (20 g) eluting with 40-60% EtOAc in pentane and then 100% EtOAc to afford a cream solid.

Yield: 1.59 g (86%)

LC-MS (Method 3): Rt 3.06 min, m/z 553 [MNa]+

### INTERMEDIATE 7

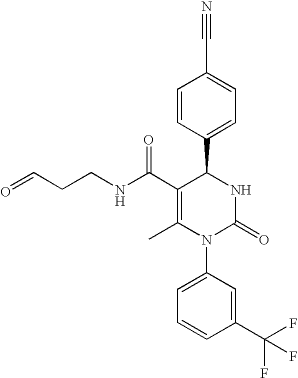

A solution of Intermediate 6 (1.59 g, 3.00 mmol) in THF (20 ml) was treated with 1M HCl (20 ml). The solution was allowed to stand at RT for 15 min before reduction of the THF in vacuo. The mixture was diluted with water (100 ml) and the product was extracted with EtOAc (150 ml). The organic layer was separated, washed with water (100 ml) and brine (50 ml), dried (Na₂SO₄) and evaporated. Chromatography using an Isolute SPE Si II cartridge (20 g) and eluting with 60-100% EtOAc in pentane gave the aldehyde as a white foam.

Yield: 820 mg (60%)

LC-MS (Method 2): Rt=2.96 min, m/z 457 [MH]+

### INTERMEDIATE 8

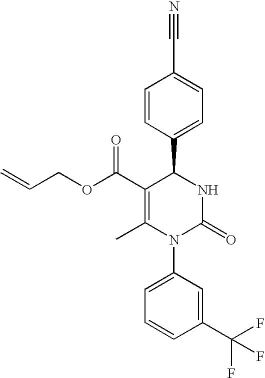

Intermediate 4 (5.00 g, 12.47 mmol) was dissolved in toluene (250 ml) and oxalyl chloride (1.30 ml) was added. The reaction mixture was stirred whilst a catalytic amount of DMF (25 drops) was added. After stirring for 1 h, allyl alcohol (1.81 ml, 31.18 mmol) was added and the reaction mixture was stirred for a further 2.5 h. The solvent was removed and the residue was dissolved in EtOAc (300 ml). The solution was washed with sat. aqueous NaHCO₃ (200 ml), water (200 ml) and brine (100 ml), dried (Na₂SO₄) and evaporated to give a pale yellow foam.

Yield: 4.80 g (87%)

LC-MS (Method 4): Rt 3.77 min, m/z 442 [MH]+

### INTERMEDIATE 9

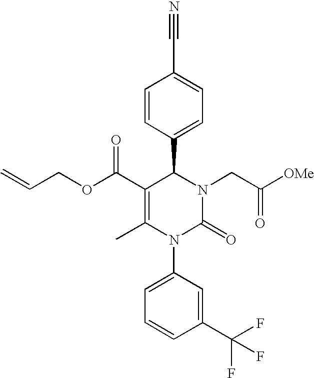

Intermediate 8 (730 mg, 1.655 mmol) was dissolved in DMF (15 ml) and the solution was cooled to −10° C. under argon. Sodium hydride (60% dispersion in mineral oil) (99 mg, 2.48 mmol) was added and the reaction mixture was stirred for 10 min before the addition of methyl bromoacetate (279 mg, 1.821 mmol). After stirring for 1 h, the reaction was quenched by the addition of sat. aqueous NH₄Cl (20 ml). The mixture was extracted with EtOAc (2×150 ml) and the combined organic extracts were washed with brine (100 ml), dried (Na₂SO₄) and evaporated. The crude product was purified on an Isolute SPE Si II cartridge (10 g) eluting with pentane and then 30-35% EtOAc in pentane. The product was obtained as a white foam.

Yield: 612 mg (72%)

LC-MS (Method 4): Rt 4.00 min, m/z 514 [MH]+

### INTERMEDIATE 10

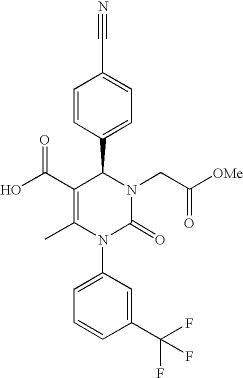

Intermediate 9 (612 mg, 1.193 mmol) and morpholine (1 ml, 11.93 mmol) were dissolved in THF (6 ml) and a catalytic amount of tetrakis(triphenylphosphine) palladium(0) (10 mg, 0.008 mmol) was added. The solution was stirred at RT under nitrogen for 1.5 h and the solvent was evaporated. The residue was dissolved in EtOAc (50 ml) and the solution was washed with 1M HCl (50 ml), water (40 ml) and brine (30 ml), dried (Na₂SO₄) and evaporated to give the product as a pale yellow foam.

Yield: 553 mg (98%)

LC-MS (Method 4): Rt 3.35 min, m/z 474 [MH]+

### INTERMEDIATE 11

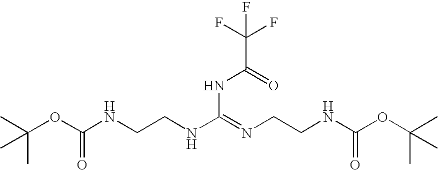

Prepared according to Bartoli, S.; Jensen, K. B.; Kilburn, J. D. J. Org. Chem.: (2003) 68, 9416-9422.

### INTERMEDIATE 12

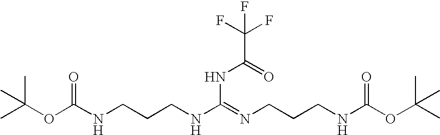

Prepared according to Bartoli, S.; Jensen, K. B.; Kilburn, J. D. J. Org. Chem.: (2003) 68, 9416-9422.

### INTERMEDIATE 13

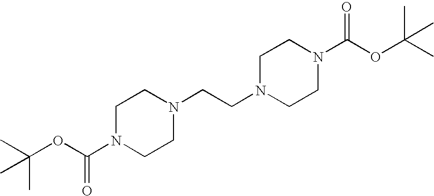

Boc-piperazine (0.5 g, 2.69 mmol), 1,2-dibromoethane (253 mg, 1.34 mmol) and NaHCO₃ (564 mg, 6.72 mmol) in acetonitrile (20 ml) were heated at 90° C. for 17 h. After cooling to RT, the solvent was removed and the residue was dissolved in EtOAc (80 ml). The organic solution was washed with water (50 ml) and brine (20 ml), dried (Na₂SO₄) and evaporated to give Intermediate 13 as a white solid.

Yield: 426 mg (80%)

LC-MS (Method 2): Rt 0.34/2.05 min, m/z 399 [MH⁺]

### INTERMEDIATE 14

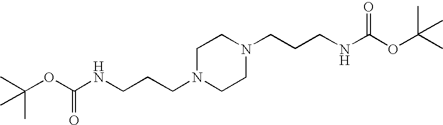

Piperazine (144 mg, 1.68 mmol), 3-(Boc-amino)propyl bromide (800 mg, 3.36 mmol) and NaHCO₃ (722 mg, 8.4 mmol) were stirred in acetonitrile (20 ml) at 90° C. for 18 h. The solvent was removed and water (30 ml) and EtOAc (60 ml) were added then the layers separated. The organic layer was dried (MgSO₄) and evaporated.

Yield: 588 mg (88%)

LC-MS (Method 2): Rt 0.35/1.84 min, 401 m/z [MH⁺]

### INTERMEDIATE 15

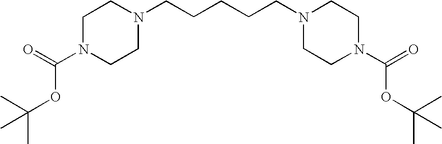

N-Boc piperazine (2.0 g, 10.75 mmol), glutaraldehyde (50% aqueous solution, 1.08 ml, 5.37 mmol) and sodium triacetoxyborohydride (3.68 g, 17.2 mmol) in DCE (30 ml) were stirred at RT under nitrogen for 3 h. The reaction mixture was quenched using sat. aqueous NaHCO₃, extracted with EtOAc, dried (MgSO₄) and evaporated to give a colourless oil which solidified on standing.

Yield: 2.00 g (42%)

LC-MS (Method 3): Rt 0.26/1.5 min, 441 m/z [MH⁺]

### INTERMEDIATE 16

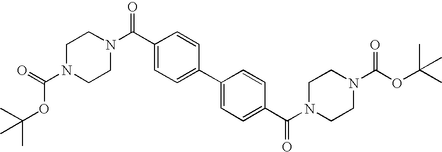

4,4′-Biphenyldicarboxylic acid (2.0 g, 8.3 mmol), N-Boc-piperazine (3.38 g, 18.2 mmol), HATU (6.55 g, 18.2 mmol) and DIPEA (9.4 ml, 55 mmol) were suspended in DMF (30 ml) and stirred at RT for 30 min. The mixture was divided into three portions and each was irradiated in the microwave at 100° C. for 5 min before the samples were re-combined, diluted with diethyl ether and filtered. The solid was washed with diethyl ether and suction dried to an off-white powder.

Yield: 3.04 g (63%)

LC-MS (Method 2): Rt 3.79 min, 579 m/z [MH⁺]

The following compounds were prepared from a di-acid and N-Boc-piperazine by a procedure similar to that used for the synthesis of Intermediate 16:

| | | | | | Rt (min) | |
|---|---|---|---|---|---|---|
| Inter- | | | Reaction | Yield | (Method | Mas [symbol] |
| mediate | Structure | Di-acid | time | (%) | 2) | [MH [symbol] |
| 17 | 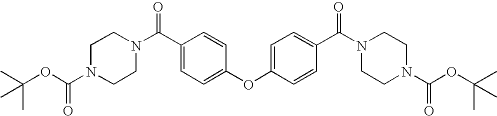 | 4,4′-Oxybis-benzoic | 30 min | 49 | *See NMR dat [symbol] | |
| 18 | 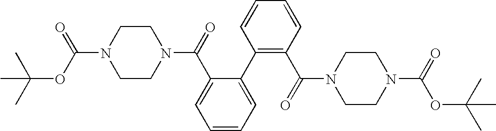 | Diphenic | 10 min(100° C.μwave) | 83 | 3.88 | 57 [symbol] |
| 19 | 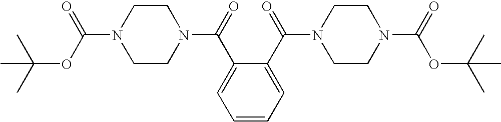 | Phthalic | 24 h | 83 | 3.40 | 52 [symbol] [M + N |
| 20 | 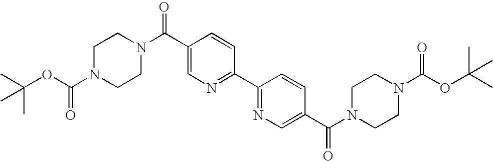 | 2,2′-Bipyridine-5,5′-dicarboxylic | 17 h | 74 | 3.19 | 58 [symbol] |
| 21 | 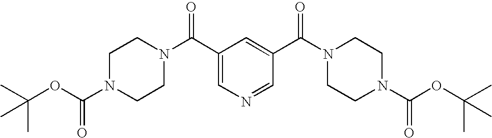 | 3,5-Pyridine-carboxylic | 17 h | 96 | 2.90 | 50 [symbol] |

*¹H-NMR (400 MHz, MeOD): δ = 7.47 (1 H, d); 7.11 (1 H, d); 3.32-3.60 (16 h, br s); 1.41 (18 H, s) [symbol]
indicates data missing or illegible when filed

### INTERMEDIATE 22

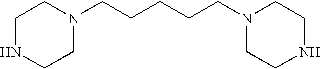

Intermediate 15 (1.0 g, 2.27 mmol) was dissolved in DCM (4 ml) and TFA (4 ml) was added. The mixture was stirred at RT for 30 min. The solvent was removed and the residue was taken up into 1:1 DCM/MeOH and applied to an SCX-2 cartridge. After washing with 1:1 DCM/MeOH then MeOH, the product was eluted with 2M NH₃ in MeOH. The solvent was removed.

Yield: 676 mg (100% (contains solvent))

LC-MS (Method 2): Rt 0.26 min, 241 m/z [MH⁺]

The following compounds were prepared using a procedure similar to that used for the synthesis of Intermediate 22:

| | | From | | Rt (min) | |
|---|---|---|---|---|---|
| Inter- | | Inter- | Yield | (Method | Mass |
| mediate | Structure | mediate | (%) | 2) | [MH]⁺ |
| 23 | 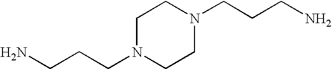 | 14 | 98 | 0.25 | 201 |
| 24 | 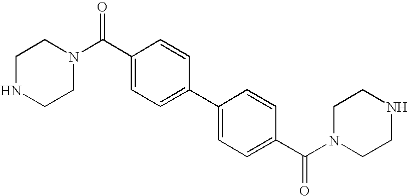 | 16 | 100 | 0.33 | 379 |
| 25 | 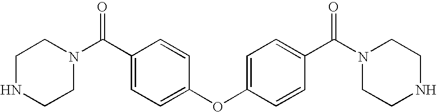 | 17 | 70 | 0.35 | 395 |
| 26 | 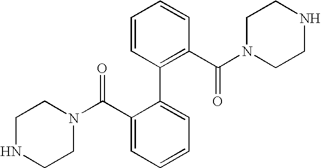 | 18 | 100 | 0.33 | 379 |
| 27 | 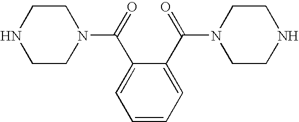 | 19 | 91 | 0.28 | 303 |
| 28 | 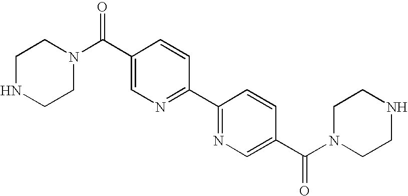 | 20 | | 0.23 | 381 |
| 29 | 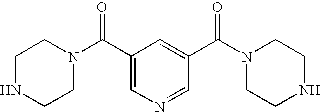 | 21 | 100 | 0.19 | 304/607 |
| 30 | 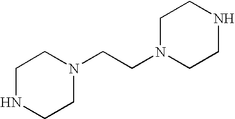 | 13 | | 0.27 | 199 |

### EXAMPLE 1

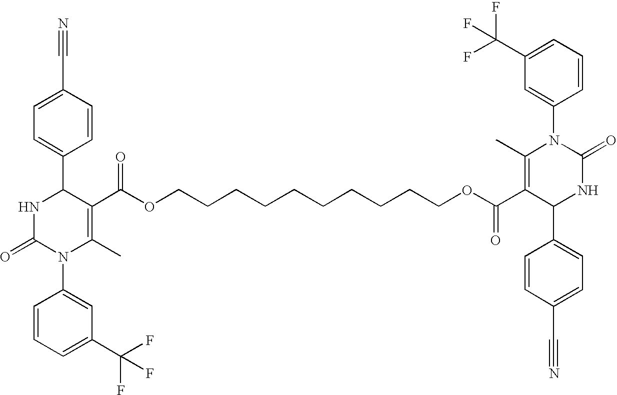

Intermediate 3 (200 mg, 0.499 mmol), DMAP (65 mg, 0.533 mmol), EDCI (96 mg, 0.503 mmol), and 1,10-decanediol (39 mg, 0.244 mmol) were dissolved in DCM (2 ml) and the solution was stirred for 17 h. The solvent was removed and the mixture was purified by HPLC (System 2).

Yield: 42 mg (18%)

LC-MS (Method 1): Rt 15.45, m/z 941.13 [MH]+

### EXAMPLE 2

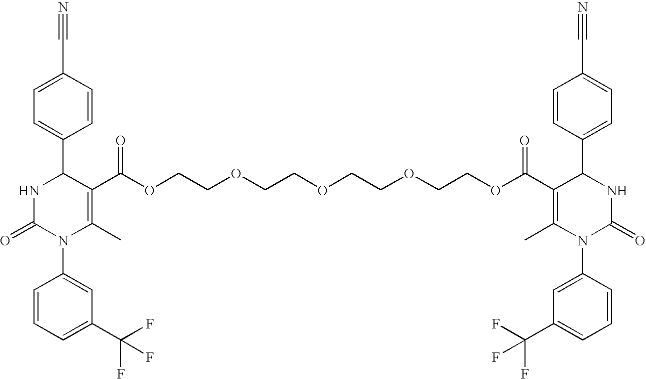

Example 2 was prepared from intermediate 3 (100 mg) and tetra(ethylene) glycol by a similar procedure to that used in Example 1, and purified using HPLC (System 2).

Yield: 62 mg (56%)

LC-MS (Method 1): Rt 12.80 min, m/z 961.17 [MH+]

### EXAMPLE 3

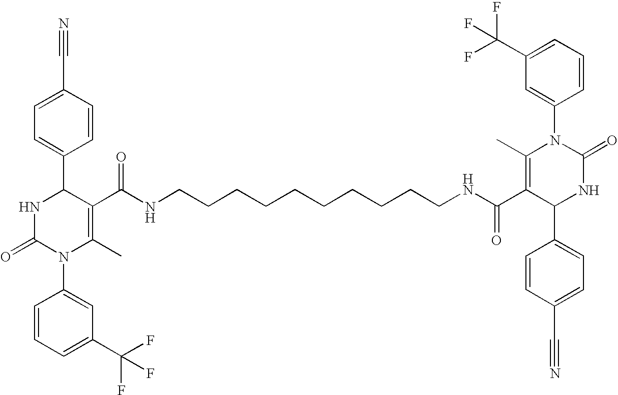

A solution of intermediate 3 (200 mg, 0.499 mmol), 1,10-diaminodecane (39 mg, 0.227 mmol), DIPEA (87 μl, 0.500 mmol), and HATU (190 mg, 0.500 mmol) in acetonitrile (2 ml) was stirred at RT for 17 h. The solvent was removed and the mixture was purified by HPLC (System 2).

Yield: 67 mg (26%)

LC-MS (Method 1): Rt 12.64, m/z 939.30 [MH]+

### EXAMPLE 4

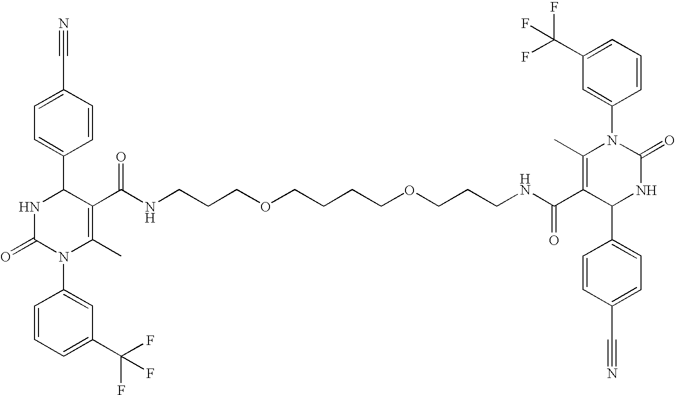

Example 4 was prepared from Intermediate 3 and 4,9-dioxa-1,12-dodecanediamide by a similar method to that used in the synthesis of Example 3.

Yield: 58%

LC-MS (Method 1): Rt 11.30, m/z 971.20 [MH]+

### EXAMPLE 5

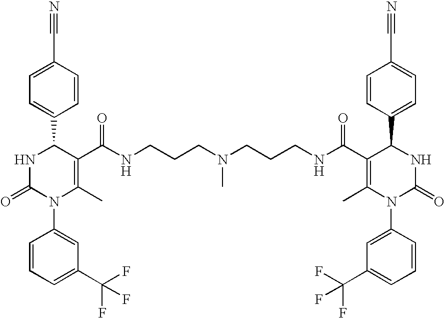

Intermediate 4 (117 g, 0.292 mmol), 3,3′-diamino-N-methyldipropylamine (21 mg, 0.146 mmol) and DIPEA (254 μl, 1.46 mmol) were dissolved in DMF (6 ml). HATU (133 mg, 0.350 mmol) was added and the solution was allowed to stand at RT for 2.5 h. The DMF was evaporated and the residue was dissolved in EtOAc (100 ml). The organic solution was washed with aqueous NaHCO₃ (60 ml), water (50 ml) and brine (40 ml). After drying (Na₂SO₄), the solvent was removed and the crude product was purified using HPLC (System 1).

Yield: 38 mg (14%)

LC-MS (Method 1): Rt 7.92 min, m/z 912.23 [MH⁺]

The following exaples were prepared in a similar manner:

| Ex- | | | | Rt | |
|---|---|---|---|---|---|
| am- | | Starting | Yield | (Method | Mass |
| ple | Structure | materials | (%) | 1) | [MH]+ |
| 6 | 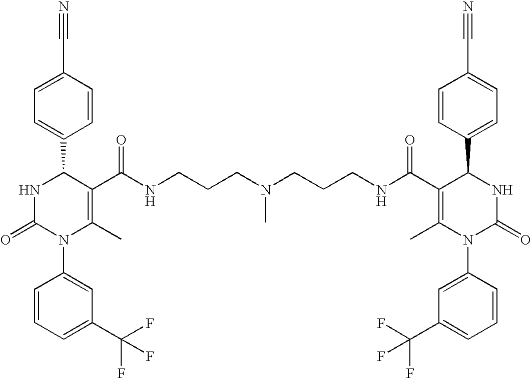 | Intermediate4 and3,3′-diamino-N-methyl-dipropylamine | 14 | 7.92 | 912.23 |
| 7 | 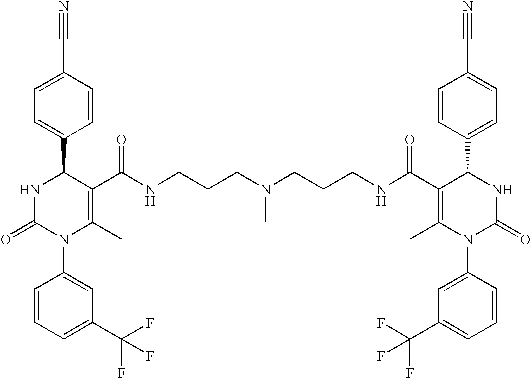 | Intermediate5 and3,3′-diamino-N-methyl-dipropylamine | 15 | 7.99 | 912.24 |
| 8 | 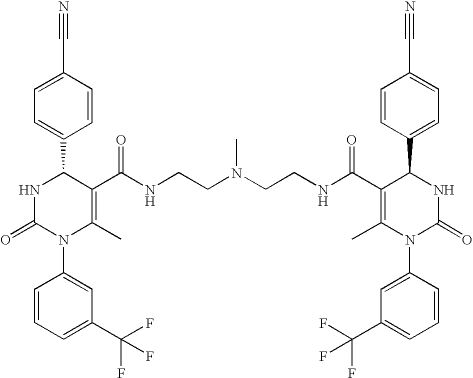 | Intermediate4 and N-methyl-2,2′-diamino-ethylamine | 6 | 7.96 | 884.19 |
| 9 | 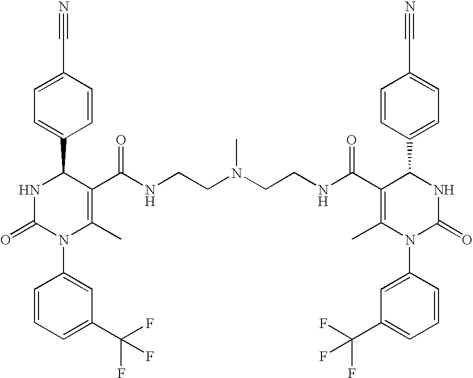 | Intermediate5 and N-methyl-2,2′-diamino-ethylamine | 15 | 8.03 | 884.14 |
| 10 | 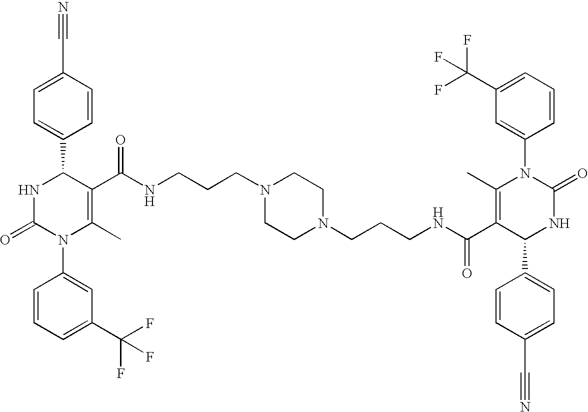 | Intermediate4 andIntermediate23 | 13 | 7.62 | 967.08 |
| 11 | 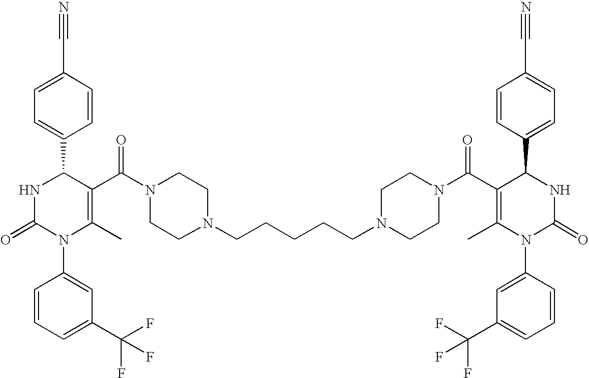 | Intermediate4 andIntermediate22 | 7 | 6.62 | 1007.18 |
| 12 | 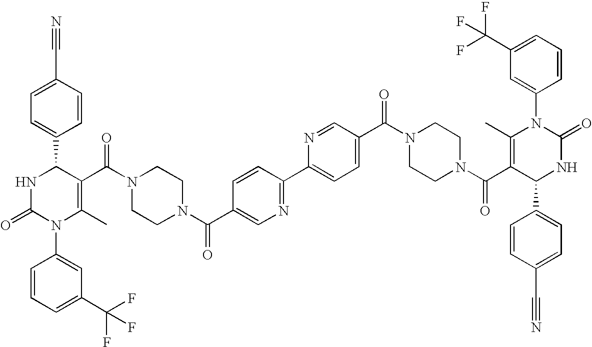 | Intermediate4 andIntermediate28 | 32 | 10.10 | 1147.15 |
| 13 | 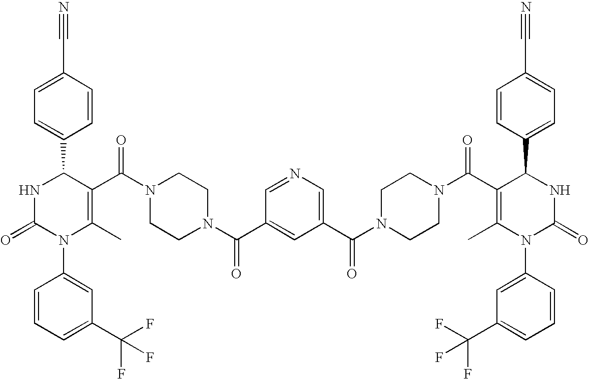 | Intermediate4 andIntermediate29 | 14 | 9.62 | 1070.17 |
| 14 | 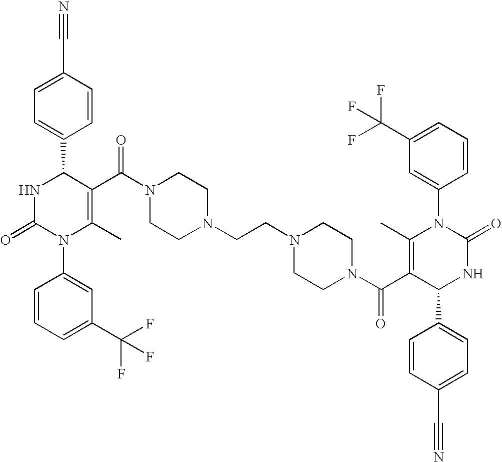 | Intermediate4 andIntermediate30 | 13 | 7.90 | 965.21 |

### EXAMPLE 15

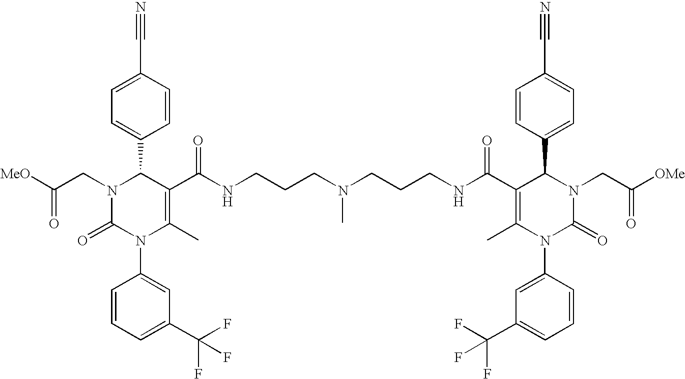

Example 15 was prepared from Intermediate 10 and 3,3′-diamino-N-methyldipropylamine using a procedure similar to that used in the synthesis of Example 5. The crude product was dissolved in MeOH and loaded onto an SCX-2 cartridge (10 g) which had been pre-treated with MeOH. The cartridge was flushed with MeOH and then the product was eluted with 2M NH₃ in MeOH. Intermediate 15 was obtained as a pale yellow foam.

Yield: 30%

LC-MS (Method 4): Rt 3.83 min, m/z 1056 [MH]+

### EXAMPLE 16

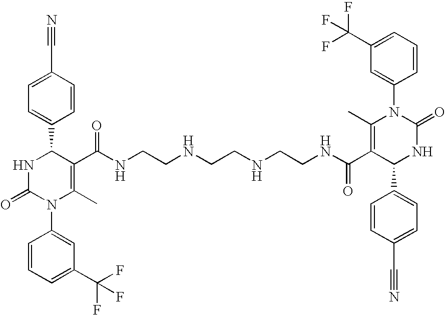

Example 16 was prepared from Intermediate 4 and triethylenetetramine using a similar procedure to that used in the synthesis of Example 5. The crude product was purified on an Isolute SPE Si II cartridge (10 g) eluting with 0-50% MeOH in EtOAc and isolated as a white solid. A small sample was further purified using HPLC (System 1).

Yield: 21%

LC-MS (Method 1): 6.89 min, m/z 913.05 min

### EXAMPLE 17

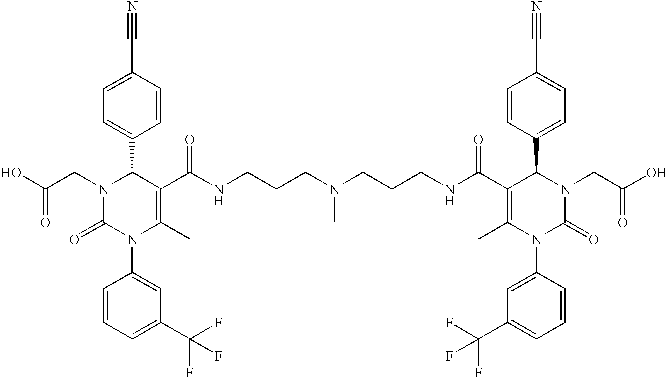

Example 15 (212 mg, 0.201 mmol) was treated with 1M NaOH (25 ml) and MeOH (15 ml). The reaction mixture was stirred at RT for 1.5 h. The mixture was acidified using 1M HCl (50 ml) and extracted with EtOAc (3×60 ml). The organic extracts were combined, washed with brine (50 ml), and dried (Na₂SO₄). Evaporation gave the diacid as a white solid.

Yield: 115 mg (56%)

LC-MS (Method 2): Rt 2.73 min, m/z 1028 [MH]+

### EXAMPLE 18

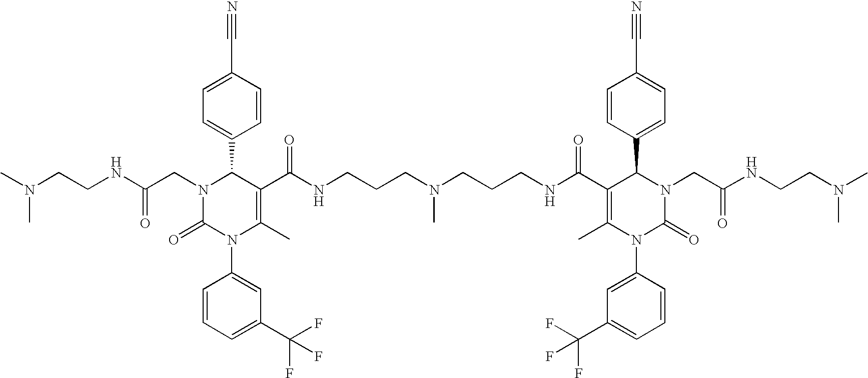

Example 18 was prepared from Example 17 and 4 equivalents of 3,3′-diamino-N-methyldipropylamine using conditions to those used in the synthesis of Example 5. Purification was achieved using HPLC (System 1) and example 18 was obtained as a white solid.

Yield: 13%

LC-MS (Method 1): Rt 5.78 min, m/z 1168.33 [MH]+

### EXAMPLE 19

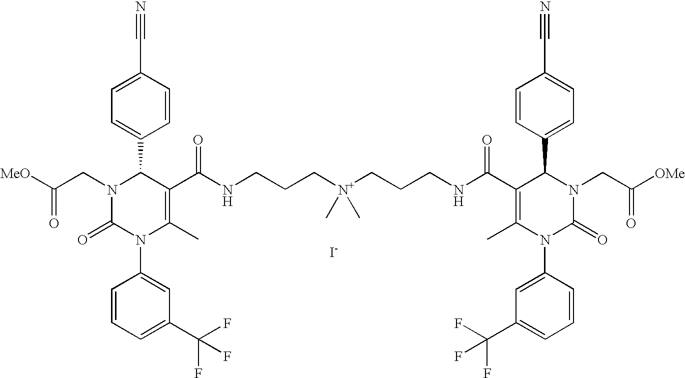

Example 15 (150 mg, 0.142 mmol) was dissolved in DCM (20 ml) and iodomethane (5 ml) was added. The solution was allowed to stand at RT for 60 h. The volatiles were evaporated.

Yield: quantitative

LC-MS (Method 4): Rt 2.86 min, m/z 1070 [M]+

### EXAMPLE 20

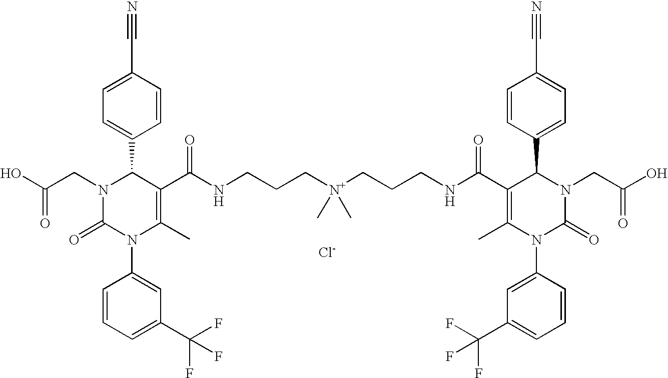

Example 20 was prepared from example 19 using a similar method to that used in the synthesis of example 17. Purification was achieved by trituration with Et₂O/DCM (5:1) giving the diacid as a pale yellow solid.

Yield: 80%

LC-MS (Method 4): Rt 2.74 min, m/z 1042 [M]+

### EXAMPLE 21

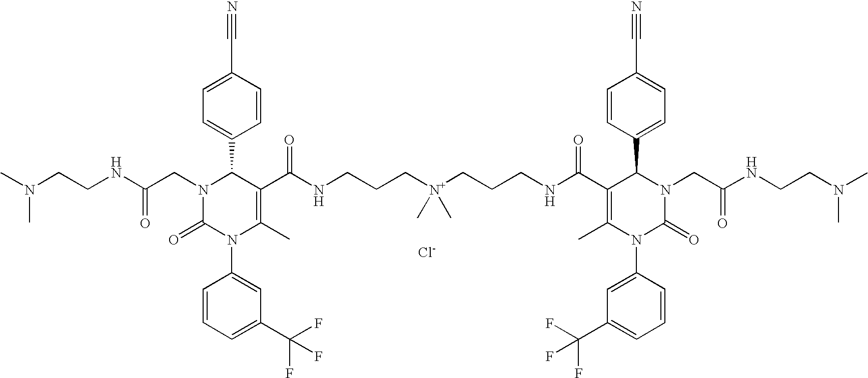

Example 21 was prepared from Example 20 and 4 equivalents of 3,3′-diamino-N-methyldipropylamine using conditions to those used in the synthesis of Example 5. Purification was achieved using HPLC (System 1) and example 21 was obtained as a pale cream solid.

Yield: 4%

LC-MS (Method 1): Rt 5.96 min, m/z 1182.07 [M]+

### EXAMPLE 22

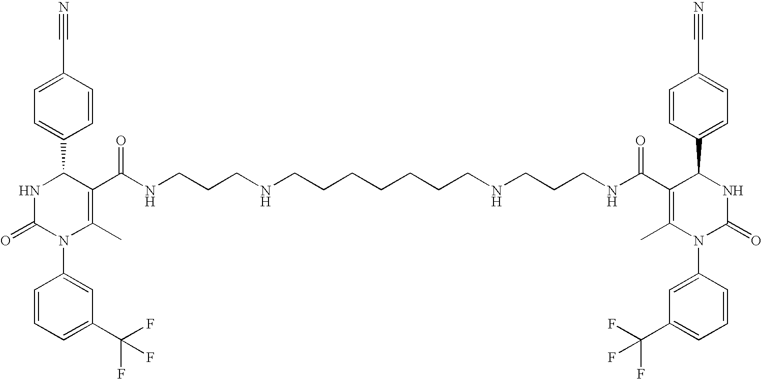

Intermediate 7 (82 mg, 0.180 mmol) and 1,7-diaminoheptane (11 mg, 0.090 mmol) were dissolved in DCE (4 ml). To the solution was added 4 Å molecular sieves and sodium triacetoxyborohydride (43 mg, 0.203 mmol), and the reaction was stirred at RT for 1 h. The mixture was diluted with DCE (20 ml) and filtered. Evaporation gave a residue; which was purified by HPLC (System 1) giving a white solid.

Yield: 10 mg (5%)

LC-MS (Method 1): Rt 6.96 min, m/z 1011.36 [MH]+

The following examples were prepared in a similar manner from Intermediate 7 and the diamine indicated:

| Ex- | |
|---|---|
| am- | |
| ple | Structure |
| 23 | 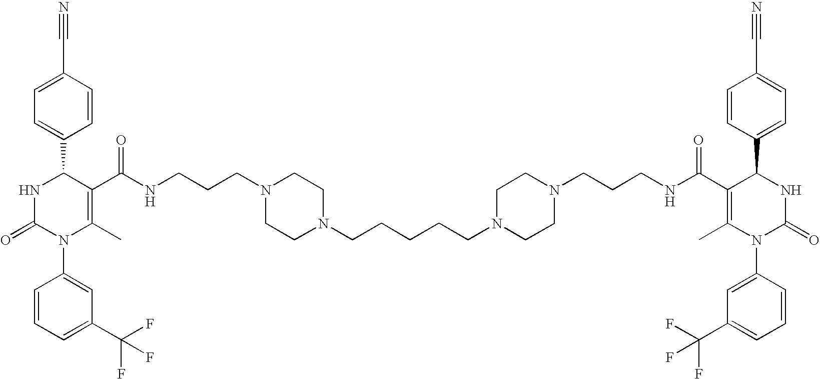 |
| 24 | 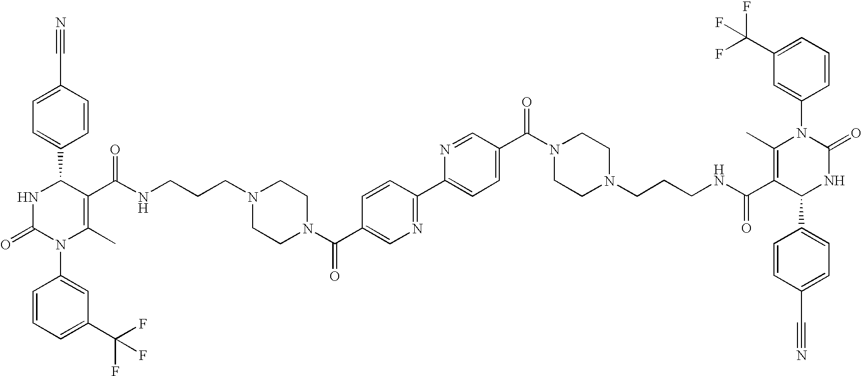 |
| 25 | 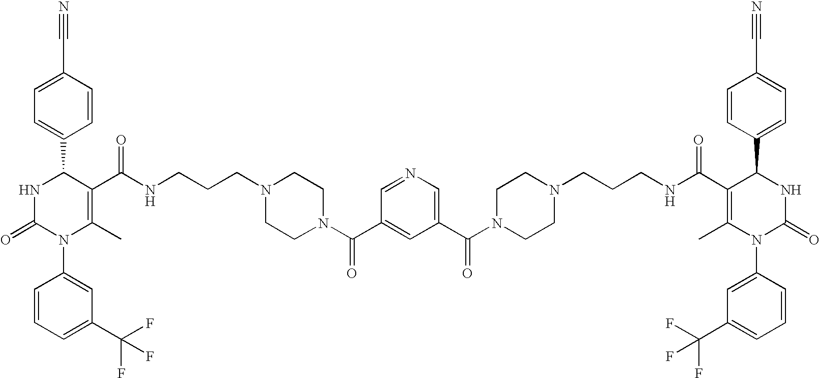 |
| 26 | 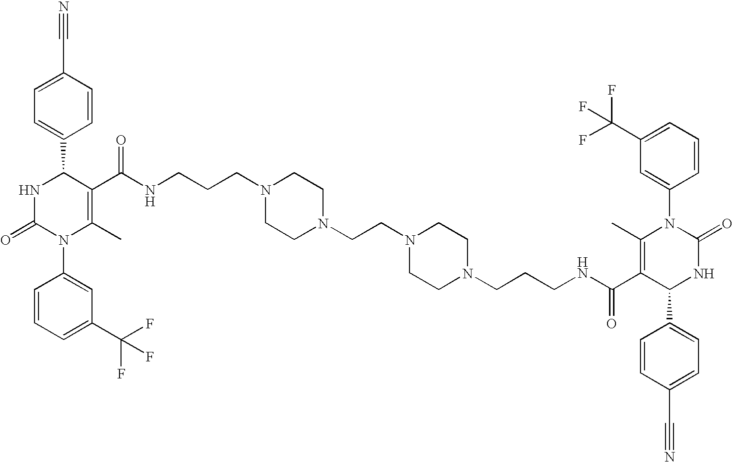 |
| 27 | 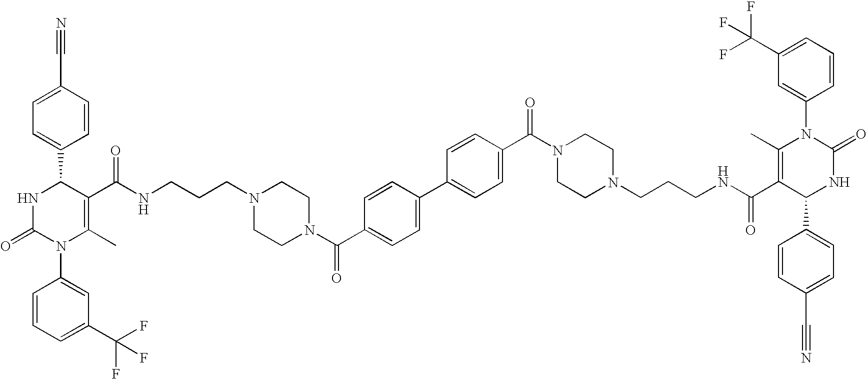 |
| 28 | 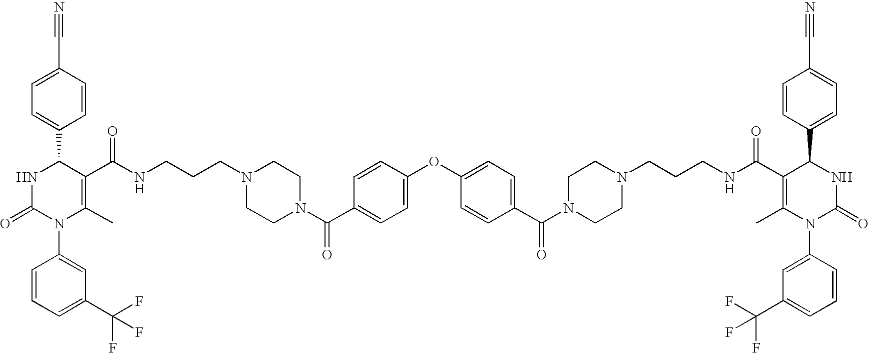 |
| 29 | 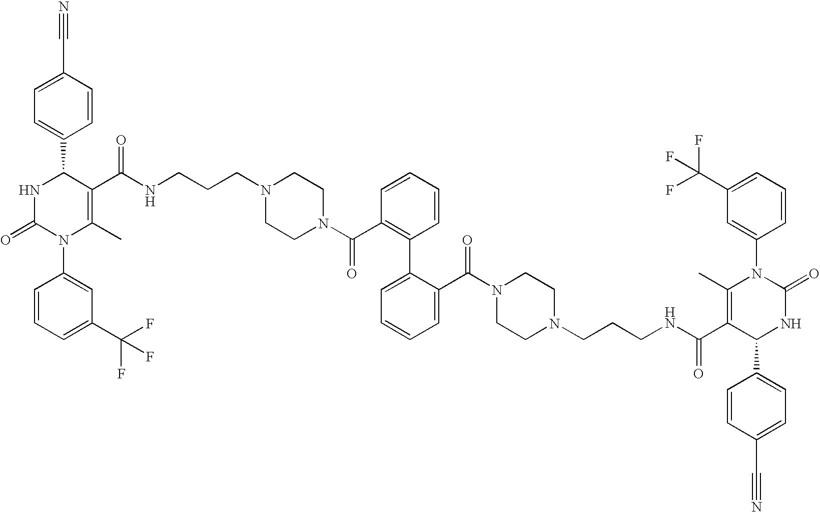 |
| 30 | 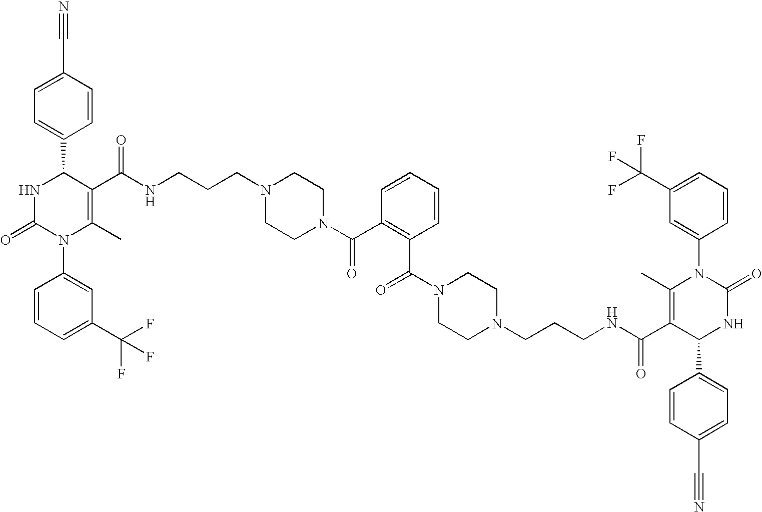 |

| Ex- | From | | | |
|---|---|---|---|---|
| am- | Diamine | Yield | Rt | Mass |
| ple | Intermediate | (%) | (Method 1) | [MH]+ |
| 23 | 22 | 11 | 6.36 | 1121.31 |
| 24 | 28 | 4 | 6.90 | 1261.29 |
| 25 | 29 | 23 | 6.70 | 1184.35 |
| 26 | 30 | 14 | 6.50 | 1079.34 |
| 27 | 24 | 28 | 7.03 | 1259.47 |
| 28 | 25 | 9 | 7.12 | 1275.42 |
| 29 | 26 | 19 | 7.06 | 1259.38 |
| 30 | 27 | 7 | 6.91 | 1183.37 |

### EXAMPLE 31

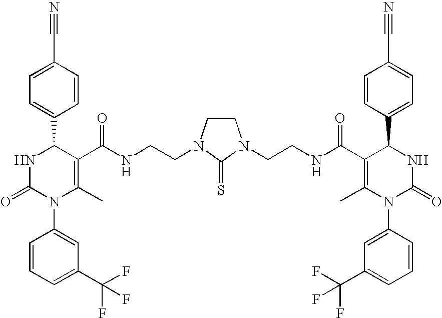

Example 16 (102 mg, 0.112 mmol) was dissolved in DCM (10 ml) and 1,1′-thiocarbonyldipyridone (13 mg, 0.0559 mmol) was added. The solution was heated under reflux for 4 h and then allowed to stand at RT for 3 days. The DCM was evaporated and the residue was chromatographed on an Isolute SPE Si II cartridge (5 g) eluting with 0-10% MeOH in EtOAc. The product-containing fractions were combined and eluted through an Isolute SPE SCX-2 cartridge, flushing with MeOH. Evaporation gave a white solid.

Yield: 66 mg (78%)

LC-MS (Method 4): Rt 3.45 min, m/z 955 [MH]+

### EXAMPLE 32

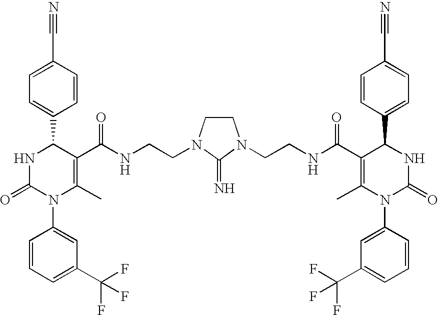

Example 31 (66 mg, 0.0691 mmol) and iodomethane (2 ml) were dissolved in IMS (6 ml) and the solution was allowed to stand at RT for 24 h. The volatiles were evaporated and the residue was dissolved in 2M NH₃ in EtOH (5 ml). The solution was heated at 50° C. for 2 h, after which time the solvent was evaporated and the product was purified using HPLC (System 1). Example 32 was obtained as a white solid.

Yield: 12 mg (19%)

LC-MS (Method 1): Rt 8.02 min, m/z 938.05 [MH]+

### EXAMPLE 33

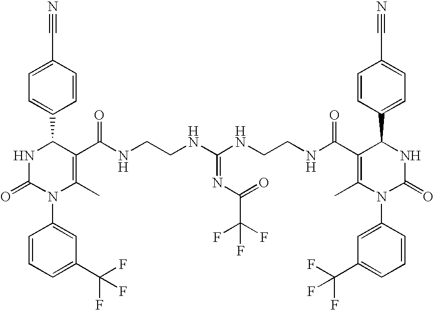

Intermediate 11 (181 mg, 0.411 mmol) was dissolved in 20% TFA in DCM (20 ml) and the solution was allowed to stand for 3 h before toluene (50 ml) was added and the volatiles were evaporated. The residue was dissolved in DMF (20 ml) and Intermediate 4 (329 mg, 0.903 mmol), DIPEA (1 ml) and HATU (343 mg, 0.903 mmol) were added. The solution was allowed to stand at RT for 1 h before evaporation of the solvent. The residue was dissolved in EtOAc (100 ml). The organic solution was washed with aqueous NaHCO₃ (60 ml), water (50 ml) and brine (40 ml). After drying (Na₂SO₄), the solvent was removed and the residual gum was triturated with diethyl ether. Further purification was achieved by chromatography on an Isolute SPE Si II cartridge (10 g) eluting with EtOAc then 2-4% MeOH in EtOAc, and example 33 was obtained as a white solid.

Yield: 190 mg (46%)

LC-MS (Method 3): Rt 3.44 min, m/z 1008 [MH]+

### EXAMPLE 34

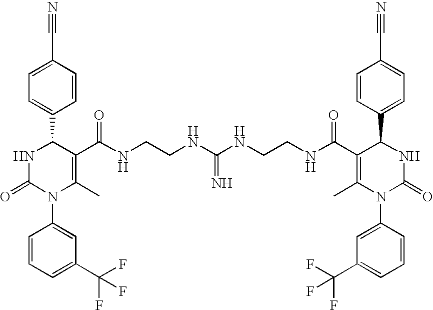

A solution of Example 33 (190 mg, 0.189 mmol) in MeOH (10 ml) was treated with a solution of potassium carbonate (261 mg, 1.89 mmol) in water (4 ml). After stirring for 30 min the solution was diluted with water (70 ml) and extracted with EtOAc (100 ml). The organic extract was dried (Na₂SO₄) and evaporated. Purification was achieved using HPLC (System 1) giving a white solid.

Yield: 93 mg (54%)

LC-MS (Method 1): Rt 8.07 min, m/z 912.21 [MH]+

### EXAMPLE 35

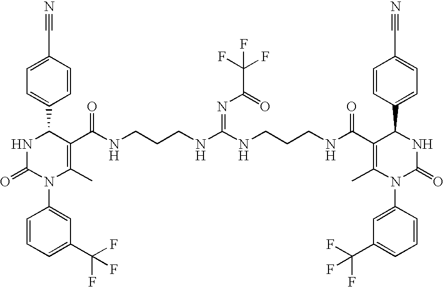

Example 35 was prepared from Intermediate 4 and Intermediate 12 by a method similar to that used in the synthesis of Example 33.

Yield: 70%

LC-MS (Method 4): Rt 3.61 min, m/z 1036 [MH]+

### EXAMPLE 36

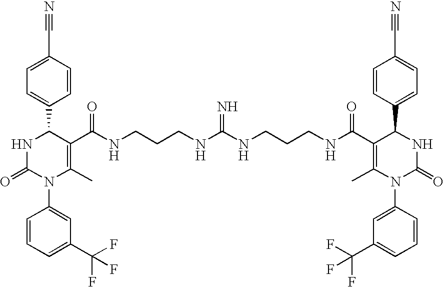

Example 36 was synthesized from Example 35 using a procedure similar to that used in the preparation of Example 34. Purification was achieved using HPLC (System 1).

Yield: 28%

LC-MS (Method 1): Rt 7.93 min, m/z 940.15 [MH]+

### EXAMPLE 37

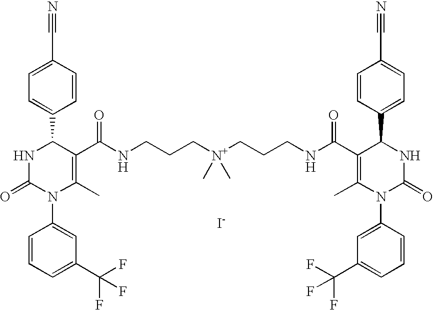

Example 6 (131 mg, 0.144 mmol) was dissolved in DCM (15 ml) and iodomethane (4 ml) was added. The reaction mixture was allowed to stand for 65 h before evaporation of the volatile materials. The mixture was purified using HPLC (System 1).

Yield: 52 mg (34%)

LC-MS (Method 1): Rt 7.88 min, m/z 926.07 [M]+

### EXAMPLE 38

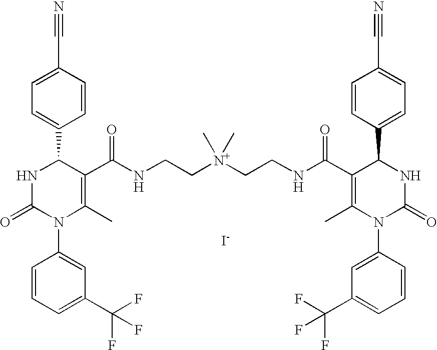

Example 38 was synthesized from Example 8 using a procedure similar to that used in the preparation of Example 37.

Yield: 31%

LC-MS (Method 1): Rt 7.98 min, m/z 898.33 [M]+

### Elastase Inhibition Assays

Various compounds of the invention were tested for their inhibitory activity towards HNE.

### Fluorescent Peptide Substrate

Assays were performed in 96-well plates at a total assay volume of 100 μl. The final concentration of the enzyme (human leukocyte elastase, Sigma E8140) was 0.00036 units/well. A peptide substrate (MeO-Suc-Ala-Ala-Pro-ValAMC, Calbiochem #324745) was used, at the final concentration of 100 μM. The final concentration of DMSO was 1% in the assay buffer (0.05M Tris.HCl, pH 7.5, 0.1M NaCl; 0.1M CaCl2; 0.0005% brij-35).

The enzymatic reaction was started by adding the enzyme. The enzymatic reaction was performed at RT and after 30 mins stopped by adding 50 μl soybean trypsin inhibitor (Sigma T-9003) at a final concentration of 50 μg/well. Fluorescence was read on the FLEXstation (Molecular Devices) using 380 nm excitation and 460 nm emission filters. The potency of the compounds was determined from a concentration series of 10 concentrations in range from 1000 nM to 0.051 nM. The results are means of two independent experiments, each performed in duplicate.

The compounds tested were shown to have IC₅₀ values for HNE in the range 1-1000 nM.

### Using Fluorescently Labelled Elastin

Assays were performed in 96-well plate at a total assay volume of 100 μl. The final concentration of the enzyme (human leukocyte elastase, Sigma E8140) was 0.002 units/well. Fluorescently labelled, solubilised elastin from bovine neck ligament (Molecular Probes, E-12056) was used at the final concentration of 15 μg/ml. The final concentration of DMSO was 2.5% in the assay buffer (0.1M Tris-HCL, pH8.0, containing 0.2 mM sodium azide).

The enzymatic reaction was started by adding the enzyme. The enzymatic reaction was performed at RT and read after 120 minutes. Fluorescence was read on the FLEXstation (Molecular Devices) using 485 nm excitation and 530 nm emission filters. The potency of the compounds was determined from a concentration series of 10 concentrations in range from 25000 nM to 1 nM. The results are means of two independent experiments, each performed in duplicate.

The compounds tested were shown to have IC₅₀ values for HNE in the range 1-1000 nM.

### Elastase Selectivity Assays

Selectivity for elastase inhibition was determined by testing the compounds against a panel of 6 proteases: plasmin, thrombin, cathepsin G, proteinase 3, trypsin, chymotrypsin (all sourced from Sigma, cat. No. P1867, T1063, C4428, P0615, T6424, C8949 respectively). Assays were performed in 96-well plate at a total assay volume of 100 μl. A common, generic substrate was used for all proteases: fluorescently labelled casein (Molecular Probes, E-6639), at the final concentration of 20 μg/ml (Cathepsin G and Chymotrypsin), 10 μg/ml (Plasmin and Thrombin) or 5 μg/ml (Proteinase 3 and Trypsin). The final concentration of the substrate was close to the respective Kₘ values as determined for this substrate. The final concentration of DMSO was 5% in the assay buffer (0.05M Tris.HCl, pH 7.5, 0.1M NaCl; 0.1M CaCl2; 0.0005% brij-35). The enzymatic reaction was started by adding the enzyme. The enzymatic reaction was performed at RT for 60 min. Fluorescence was read on the FLEXstation (Molecular Devices) using 589 nm excitation and 617 nm emission filters. The potency of the compounds was determined from a concentration series of 8 concentrations in range from 500 μM to 0.2 μM. The results are means of two independent experiments, each performed in duplicate.

The compounds tested showed selectivities for a range of proteases from 1 to >300 fold.

### Membrane Bound Elastase

Blood was collected from healthy human volunteers. PMNs were isolated by density centrifugation on ficol and red blood cells lysed hypotonically Cells were fixed with paraformaldehyde/gluteraldehyde and washed by centrifugation.

Compounds were made up in HBSS containing and incubated for 5 minutes at 37° C. with cells. Fluorogenic AAPV substrate (Calbiochem #324745) was added to each well to make 100 μl final volume and the plate read using a Spectramax Gemini Ex 380 nm Em 460 for 30 minutes at 37° C.

Compounds of the invention examined in this assay were found to exhibit IC₅₀ values of less than 100 nM, preferably less than 20 nM.

Intracellular Elastase (Controlled with Lysed Cell Elastase)

PMNs were isolated as described previously. PMNs were added to 96-well polypropylene plates and DMSO or compound added to each well to give 150 μl volume. The plate was incubated at 37° C. for 30 minutes. Cells were washed by centrifugation and lysed with HBSS containing 0.04% triton. Cell debris was pelleted and the supernatant transferred to a fresh pate, with compounds or DMSO. Fluorogenic MPV substrate was added to all wells and the plate was read using a Spectramax Gemini Ex 380 nm Em 460 for 30 minutes at 37° C.

Neutrophil Released Elastase Activity Assay (Human, Mouse, Guinea Pig) Generation of Released Neutrophil Elastase, from Guinea Pigs

Guinea pigs were treated with an LPS aerosol. Animals were left for 4 hours, euthanized and the lungs ravaged to recover PMN. Bronchoalveolar lavage fluid (BAL) was spun at 400 g for 10 minutes and the cells resuspended in HBSS. 10 μM cytocholasin B was added to the cell suspension and incubated at 37° C. for 5 minutes after which 1 μM fMLP was added for a further 5 minutes. Cells were centrifuged at 400 g for 10 minutes. ‘Elastase rich supernatant’ was transferred to a fresh tube.

Generation of Released Neutrophil Elastase, from Mice

Mice were anaesthetised and treated with LPS aerosol. Animals were left for 4 hours, euthanized and the lungs ravaged to recover PMN. Bronchoalveolar lavage fluid (BAL) was centrifuged at 400 g for 10 minutes and the cells resuspended in 1 ml of HBSS. 10 μM cytocholasin B was added to the cell suspension and incubated at 37° C. for 5 minutes after which 1 μM fMLP was added for a further 5 minutes. Cells were centrifuged at 400 g for 10 minutes. ‘Elastase rich supernatant’ was transferred to a fresh tube.

Generation of Human Released Neutrophil Elastase, from Humans

Human PMN were isolated as described previously. 10 μM cytocholasin B was added to the cell suspension and incubated at 37° C. for 5 minutes after which 1 μM fMLP was added for a further 5 minutes. Cells were centrifuged at 400 g for 10 minutes. ‘Elastase rich supernatant’ was transferred to a fresh tube.

To a clear bottomed 96-well plate compounds were added and incubated for 5 minutes at 37)C with ‘elastase rich’ supernatant. Fluorogenic AAPV substrate was added to all wells and the plate read using a Spectramax Gemini Ex 380 nm Em 460 for 30 minutes at 37° C. For comparison, an activity matched control of human elastase was also run.

### HNE Induced Lung Hemorrhage in the Rat

Instillation of human neutrophil elastase (HNE) into rat lung causes acute lung damage. The extent of this injury can be assessed by measuring lung hemorrhage. Male Sprague Dawley rats (175-220 g) were obtained from Harlan UK Ltd., full barrier-bred and certified free from specified micro-organisms on receipt. Animals were weighed and randomly assigned to treatment groups (7-12 animals per group).

The vehicle used was 1% DMSO/Saline. Inhibitors were dissolved in 1% DMSO before the addition of 0.9% saline.

Animals in each study used to determine the efficacy of the elastase inhibitors delivered locally to the lung by a variety of routes. Rats were anaesthetised with the inhaled anaesthetic Isoflurane (4%) when the dose was given from 30 minutes to 6 h prior to human neutrophil elastase (HNE) administration or terminally anaesthetised with hypnorm:hypnovel:water (1.5:1:2 at 2.7 ml/kg) when the predose was given at less than 30 minutes prior to HNE administration and dosed either intratracheally (i.t.) by transoral administration using a Penn Century microsprayer or intranasally (i.n.) by dropping the fluid on to the nares. Animals either received vehicle or compound at a dose volume of 0.5 ml/kg.

Animals that had been allowed to recover after dosing were terminally anaesthetised with hypnorm:hypnovel:water (1.5:1:2 at 2.7 ml/kg). Once sufficiently anaesthetised, HNE (600 units/ml) or sterile saline was administered by transoral tracheal instillation at a volume of 10 μL using a Penn Century microsprayer. Animals were kept warm in a temperature controlled box and given top up doses of anaesthetic as required to ensure continuous anaesthesia until termination.

Animals were sacrificed (0.5 ml to 1 ml sodium pentobarbitone) one hour post HNE challenge. The trachea was exposed and a small incision made between two tracheal rings allowing a cannula (10 gauge, O.D. 2-10 mm, Portex Ltd.) to be inserted approximately 2 cm into the trachea towards the lung. This was secured into place with a cotton ligature. The lungs were then lavaged (BAL) three times with fresh 4 ml aliquots of heparinised (10 units/ml) phosphate buffered saline (PBS). The resultant BALF was kept on ice until it was centrifuged.

The BALF was centrifuged at 1000 r.p.m. for 10 minutes in a centrifuge cooled to between 4 and 10° C. The supernatant was discarded and the cell pellet resuspended in 1 ml 0.1% CETAB/PBS to lyse the cells. Cell lysates were frozen until spectrophotometric analysis for blood content could be made. Standards were prepared by making solutions of whole rat blood in 0.1% CETAB/PBS.

Once defrosted 100 μl of each lysed cell suspension was placed into a separate well of a 96 well flat bottomed plate. All samples were tested in duplicate and 100 μl 0.1% CETAB/PBS was included on the plate as a blank. The OD of the contents of each well was measured at 415 nm using a spectramax-250 (Molecular devices).

A standard curve was constructed by measuring the OD (at 415 nm) of different concentrations of blood in 0.1% CETAB/PBS (30, 10, 7, 3, 1, 0.3, 0.1 μl/ml).

The amount of blood in each experimental sample was calculated by comparison to the standard curve. Data were then analysed as below:

1) The mean OD for duplicates was calculated
2) The value for the blank was subtracted from the value for all other samples
3) Data were assessed to evaluate the normality of distribution.

The compounds were shown to have desirable HNE inhibitory activity.

1. A compound of formula (I)
M-L-M (I)
wherein L is a linker and each M is independently a group of formula (II): 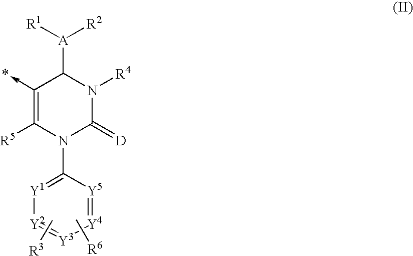
wherein
A is aryl or heteroaryl;
D is oxygen or sulphur;
R¹, R² and R³ are independently each hydrogen, halogen, nitro, cyano, alkyl, hydroxy or alkoxy, wherein alkyl and alkoxy can be further substituted with one to three identical or different radicals selected from the group consisting of halogen, hydroxy and alkoxy;
R⁴ is hydrogen, alkyl, alkylcarbonyl, alkoxycarbonyl, alkenoxycarbonyl, hydroxycarbonyl, aminocarbonyl, arylcarbonyl, heteroarylcarbonyl, heterocycloalkylcarbonyl, heteroaryl, heterocycloalkyl or cyano, wherein alkylcarbonyl, alkoxycarbonyl, and aminocarbonyl can be further substituted with one to three identical or different radicals selected from the group consisting of cycloalkyl, hydroxy, alkoxy, alkoxycarbonyl, hydroxycarbonyl, aminocarbonyl, cyano, amino, heteroaryl, heterocycloalkyl and tri-(alkyl)-silyl, and wherein heteroarylcarbonyl, heterocycloalkylcarbonyl, heteroaryl and heterocycloalkyl can be further substituted with alkyl; or
R⁴ represents a group of Formula (VIII) 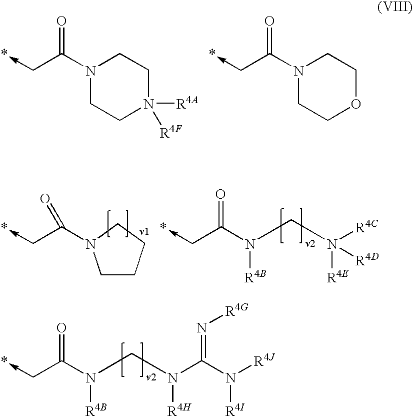
wherein
R^{4A}, R^{4B}, R^{4D}, R^{4E}, R^{4G}, R^{4H}, R^{4I} and R^{4J} are independently hydrogen or alkyl, or R^{4H} and R^{4I} may be joined together with the nitrogen atom to which they are attached to form a ring;
R^{4F} is a lone pair or R^{4F} is alkyl and the nitrogen atom to which it is attached is quaternary and carries a positive charge;
R^{4C} is a lone pair or R^{4C} is alkyl and the nitrogen atom to which it is attached is quaternary and carries a positive charge; or any two of R^{4C}, R^{4D} or R^{4E} may be joined together with the nitrogen atom to which they are attached to form a ring, optionally containing a further heteroatom selected from oxygen or nitrogen;
v1 is 1-3;
v2 is 1-6;
R⁵ is alkyl, which can be substituted with one to three identical or different radicals selected from the group consisting of halogen, hydroxy, alkoxy, alkenoxy, alkylthio, amino, hydroxycarbonyl, alkoxycarbonyl and the radical —O-(alkyl)-O-(alkyl); or R⁵ is amino;
R⁶ is halogen, nitro, cyano, alkyl, hydroxy or alkoxy, wherein alkyl and alkoxy can be further substituted with one to three identical or different radicals selected from the group consisting of halogen, hydroxy and alkoxy; and
Y¹, Y², Y³, Y⁴ and Y⁵ are independently each C or N, with the proviso that the ring in which they are comprised contains no more than 2 N atoms
or a pharmaceutically acceptable salt, solvate or N-oxide thereof.

2. The compound according to claim 1, wherein A is phenyl.

3. The compound according to claim 1, wherein the groups M have the stereochemistry shown below;

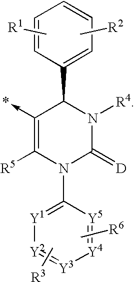

4. The compound according to claim 1, wherein R¹ is hydrogen.

5. The compound according to claim 1, wherein R² is —CN.

6. The compound according to claim 1, wherein R³ is hydrogen.

7. The compound according to claim 1, wherein R⁴ is hydrogen.

8. The compound according to claim 1, wherein R⁴ is of Formula (VIII) as defined in claim 1.

9. The compound according to claim 1, wherein R⁵ is methyl.

10. The compound according to claim 1, wherein R⁶ is trifluoromethyl.

11. The compound according to claim 1, wherein none of Y¹, Y², Y³, Y⁴ and Y⁵ is N.

12. The compound according to claim 1, wherein D is oxygen.

13. The compound according to claim 1, which is of the formula (IV)
(M-L)ₜ-G (IV)
wherein t is 2 to 20; and
G is aryl, heteroaryl, alkyl, cycloalkyl, nitrogen, a dendrimer or a group of any of formulae (V) to (VII): 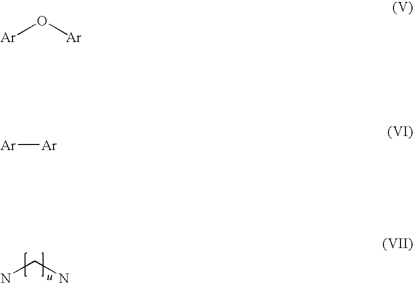
wherein
Ar is aryl or heteroaryl; and
u is 2 to 20.

14. The compound according to claim 12, wherein G is phenoxyphenyl, biphenyl, bipyridyl, ethylenediamino, propylenediamino or a dendrimer.

15. The compound according to claim 1, wherein L is a group of formula (III)
L^{a}-R⁷-L^{b}-W-L^{b}-R⁷-L^{a}- . . . 9 (III)
wherein
L^{a} is a bond or group —C(O)—;
L^{b} is a bond or group —C(O)—;
R⁷ is a bond or an alkylene or cycloalkylene group;
W is a bond or is selected from the following divalent radicals
—(O—R^{8A})ₘ₁—O—
—N(R^{9A})—(O—R^{8A})ₘ₁—R^{8A}—N(R^{9A})—
N(R^{9A})—R^{8B}—N(R^{9B})(R^{9C})—R^{8B}—N(R^{9A})—
N(R^{9A})—R^{8B}—N(R^{10B})C(═NR^{10A})(NR^{10C})—R^{8B}—N(R^{9A})—
—N(R^{9A})—R^{8B}—N(R^{9A})—
—N(R^{9A})—R^{8B}—N(R^{9A})—R^{8B}—N(R^{9A})—R^{8B}—N(R^{9A})— 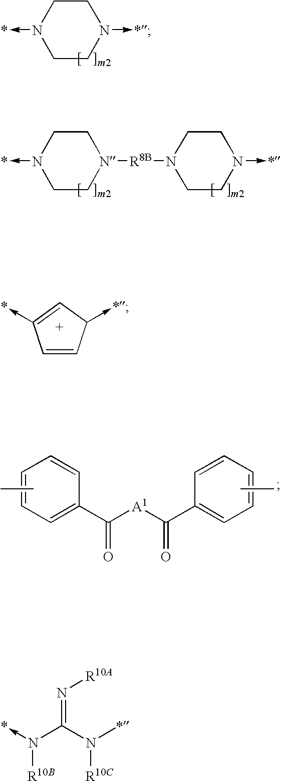
wherein
m1 is 1-4;
R^{8A} is an alkylene or cycloalkylene group;
R^{8B} is an alkylene or cycloalkylene group, or a group of Formula A²;
R^{9A} is hydrogen or alkyl;
one of R^{9B} or R^{9C} is a lone pair and the other is hydrogen or alkyl, or R^{9B} and R^{9C} are both alkyl, in which case the nitrogen to which they are attached is quaternary and carries a positive charge; or R^{9B} and R^{9C} together with the nitrogen to which they are attached form a ring;
R^{10A} is hydrogen or alkyl;
R^{10B} and R^{10C} are independently hydrogen or alkyl, or alternatively R^{10B} and R^{10C} may be joined together to form a ring;
m2 is 1-3;
A¹ is —N(R^{9A})—R⁸—N(R^{9B})(R^{9C})—R⁸—N(R^{9A})— or —N(R^{9A})—R⁸—N(R^{10B})C(═NR^{10A})(NR^{10C})—R⁸—N(R^{9A})—; and
A² is one of the following groups 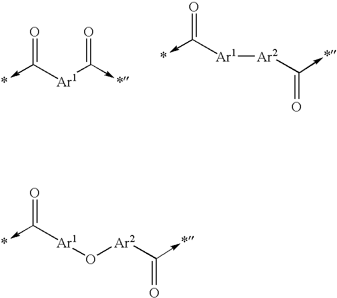
wherein Ar¹ and Ar² are each independently an aryl or heteroaryl group.

16. The compound according to claim 14, wherein each L^{a} is independently —C(O)— or a covalent bond.

17. The compound according to claim 16, wherein L^{a} is a group C(O).

18. The compound according to claim 15, wherein R⁷ and L^{b} comprise a bond.

19. The compound according to claim 15, wherein W is —N(R^{9A})—R^{8B}—N(R^{9B})(R^{9C})—R^{8B}—N(R^{9A})—.

20. The compound according to claim 15, wherein W is —N(R^{9A})—R^{8B}—N(R^{10B})C(═NR^{10A})(NR^{10C})—R^{8B}—N(R^{9A}).

21. The compound according to claim 15, wherein W is

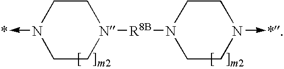

22. The compound according to claim 15, wherein W is —N(R9A)-R8B-N(R9A)-R8B-N(R9A)-R8B-N(R9A)-.

23. The compound according to claim 15, wherein W is

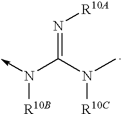

24. The compound according to claim 15, wherein W is —N(R9B)(R9C)—.

25. The compound according to claim 1, wherein A is aryl or heteroaryl;
D is oxygen or sulphur;
R¹, R² and R³ are independently each hydrogen, halogen, nitro, cyano, alkyl, hydroxy or alkoxy, wherein alkyl and alkoxy can be further substituted with one to three identical or different radicals selected from the group consisting of halogen, hydroxy and alkoxy;
R⁴ is hydrogen, alkyl, alkylcarbonyl, alkoxycarbonyl, alkenoxycarbonyl, hydroxycarbonyl, aminocarbonyl, arylcarbonyl, heteroarylcarbonyl, heterocycloalkylcarbonyl, heteroaryl, heterocycloalkyl or cyano, wherein alkylcarbonyl, alkoxycarbonyl, and aminocarbonyl can be further substituted with one to three identical or different radicals selected from the group consisting of cycloalkyl, hydroxy, alkoxy, alkoxycarbonyl, hydroxycarbonyl, aminocarbonyl, cyano, amino, heteroaryl, heterocycloalkyl and tri-(alkyl)-silyl, and wherein heteroarylcarbonyl, heterocycloalkylcarbonyl, heteroaryl and heterocycloalkyl can be further substituted with alkyl; or
R⁴ represents a group of Formula (VIII); 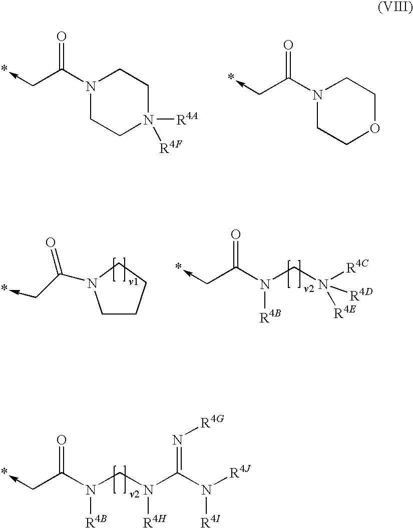
wherein
R^{4A}, R^{4B}, R^{4D}, R^{4E}, R^{4G}, R^{4H}, R^{4I} and R^{4J} are independently hydrogen or alkyl, or R^{4H} and R^{4I} may be joined together with the nitrogen atom to which they are attached to form a ring;
R^{4F} is a lone pair or R^{4F} is alkyl and the nitrogen atom to which it is attached is quaternary and carries a positive charge;
R^{4C} is a lone pair or R^{4C} is alkyl and the nitrogen atom to which it is attached is quaternary and carries a positive charge; or any two of R^{4C}, R^{4D} or R^{4E} may be joined together with the nitrogen atom to which they are attached to form a ring, optionally containing a further heteroatom selected from oxygen or nitrogen;
v1 is 1-3;
v2 is 1-6;
R⁵ is alkyl which can be substituted with one to three identical or different radicals selected from the group consisting of halogen, hydroxy, alkoxy, alkenoxy, alkylthio, amino, hydroxycarbonyl, alkoxycarbonyl and the radical —O-(alkyl)-O-(alkyl); or R⁵ is amino;
R⁶ is halogen, nitro, cyano, alkyl, hydroxy or alkoxy, wherein alkyl and alkoxy can be further substituted with one to three identical or different radicals selected from the group consisting of halogen, hydroxy and alkoxy; and
Y¹, Y², Y³, Y⁴ and Y⁵ are independently each C or N, with the proviso that the ring in which they are comprised contains no more than 2 N atoms.

26. The compound according to claim 1, as defined in any of Examples 1 to 38.

27. The compound according to claim 1, as defined in any of Examples 1 to 17.

28. The compound according to claim 1, as defined in any of Examples 7, 8, 16, 18, 21, 32, 34, 36, 37, 38.

29. (canceled)

30. A pharmaceutical composition comprising a compound of claim 1 and a pharmaceutically acceptable carrier or excipient.

31. A method for treating a condition selected from asthma, inflammatory bowel diseases, chronic obstructive pulmonary disease (COPD), chronic bronchitis, lung fibrosis, pneumonia, acute respiratory distress syndrome (ARDS), pulmonary emphysema, smoking-induced emphysema, sarcoidosis, bronchiectasis and cystic fibrosis (CF) wherein said method comprises administering, to a patient in need of such treatment, a compound according to claim 1.

32. The method according to claim 31, wherein the condition is COPD.

33. The method according to claim 31, wherein the condition is CF.

34. The method according to claim 31, wherein the condition is ulcerative colitis or Crohn's disease.

35. The method according to claim 31, wherein the condition is a respiratory condition and the compound is to be administered via the inhaled route.
